SEQUENCE LISTING (124 sequences)

SEQ ID NO: 1 - RNA, 19 nt, Artificial
  Description of Artificial Sequence; Note = Synthetic Construct
caaacaagucagucuaacg

SEQ ID NO: 2 - RNA, 19 nt, Artificial
  Description of Artificial Sequence; Note = Synthetic Construct
ccuuagaauggcuuguauu

SEQ ID NO: 3 - RNA, 19 nt, Artificial
  Description of Artificial Sequence; Note = Synthetic Construct
auuucucccacgcugacua

SEQ ID NO: 4 - RNA, 19 nt, Artificial
  Description of Artificial Sequence; Note = Synthetic Construct
uaagugagcuguggagaga

SEQ ID NO: 5 - RNA, 19 nt, Artificial
  Description of Artificial Sequence; Note = Synthetic Construct
uuuaguggaauaguugaaa

SEQ ID NO: 6 - RNA, 19 nt, Artificial
  Description of Artificial Sequence; Note = Synthetic Construct
auuucaacugacucccuaa

SEQ ID NO: 7 - RNA, 19 nt, Artificial
  Description of Artificial Sequence; Note = Synthetic Construct
caauucucuguaucuugca

SEQ ID NO: 8 - RNA, 19 nt, Artificial
  Description of Artificial Sequence; Note = Synthetic Construct
uggcaauauuuacagagaa

SEQ ID NO: 9 - RNA, 19 nt, Artificial
  Description of Artificial Sequence; Note = Synthetic Construct
guuucaugacuuaguguuu

SEQ ID NO: 10 - RNA, 19 nt, Artificial
  Description of Artificial Sequence; Note = Synthetic Construct
auaacacugacugcaugaa

SEQ ID NO: 11 - RNA, 19 nt, Artificial
  Description of Artificial Sequence; Note = Synthetic Construct
guuucaugacuuaguguuu

SEQ ID NO: 12 - RNA, 19 nt, Artificial
  Description of Artificial Sequence; Note = Synthetic Construct
auaacacugacugcaugaa

SEQ ID NO: 13 - RNA, 19 nt, Artificial
  Description of Artificial Sequence; Note = Synthetic Construct
ucaaacagacaugauauag

SEQ ID NO: 14 - RNA, 19 nt, Artificial
  Description of Artificial Sequence; Note = Synthetic Construct
cauaucaacgucuguugau

SEQ ID NO: 15 - RNA, 19 nt, Artificial
  Description of Artificial Sequence; Note = Synthetic Construct
ucaaacagacaugauauag

SEQ ID NO: 16 - RNA, 19 nt, Artificial
  Description of Artificial Sequence; Note = Synthetic Construct
cauaucaacgucuguugau

SEQ ID NO: 17 - RNA, 19 nt, Artificial
  Description of Artificial Sequence; Note = Synthetic Construct
aaauucacugcuuuguggc

SEQ ID NO: 18 - RNA, 19 nt, Artificial
  Description of Artificial Sequence; Note = Synthetic Construct
gacagcaaacagcagugaa

SEQ ID NO: 19 - RNA, 19 nt, Artificial
  Description of Artificial Sequence; Note = Synthetic Construct
guuucuuacucagacagag

SEQ ID NO: 20 - RNA, 19 nt, Artificial
  Description of Artificial Sequence; Note = Synthetic Construct
cucauuucuguaguaagac

SEQ ID NO: 21 - RNA, 19 nt, Artificial
  Description of Artificial Sequence; Note = Synthetic Construct
guuucuuacucagacagag

SEQ ID NO: 22 - RNA, 19 nt, Artificial
  Description of Artificial Sequence; Note = Synthetic Construct
cucauuucuguaguaagac

SEQ ID NO: 23 - RNA, 19 nt, Artificial
  Description of Artificial Sequence; Note = Synthetic Construct
cuuuagaugauaagcauuu

SEQ ID NO: 24 - RNA, 19 nt, Artificial
  Description of Artificial Sequence; Note = Synthetic Construct
aaaucuuucagucucaaga

SEQ ID NO: 25 - RNA, 19 nt, Artificial
  Description of Artificial Sequence; Note = Synthetic Construct
ccuguuacacagaauaaau

SEQ ID NO: 26 - RNA, 19 nt, Artificial
  Description of Artificial Sequence; Note = Synthetic Construct
auugaaauucuguguaacu

SEQ ID NO: 27 - RNA, 19 nt, Artificial
  Description of Artificial Sequence; Note = Synthetic Construct
augaaaucaaacagacaug

SEQ ID NO: 28 - RNA, 19 nt, Artificial
  Description of Artificial Sequence; Note = Synthetic Construct
caagugcugucugauucca

SEQ ID NO: 29 - RNA, 19 nt, Artificial
  Description of Artificial Sequence; Note = Synthetic Construct
aggccucggccaaacacuc

SEQ ID NO: 30 - RNA, 19 nt, Artificial
  Description of Artificial Sequence; Note = Synthetic Construct
gaguauuugugcagcgagg

SEQ ID NO: 31 - RNA, 19 nt, Artificial
  Description of Artificial Sequence; Note = Synthetic Construct
aagccauguuuccuguaau

SEQ ID NO: 32 - RNA, 19 nt, Artificial
  Description of Artificial Sequence; Note = Synthetic Construct
auuuacagagaaacauugu

SEQ ID NO: 33 - RNA, 19 nt, Artificial
  Description of Artificial Sequence; Note = Synthetic Construct
cugauuucaugacagcaag

SEQ ID NO: 34 - RNA, 19 nt, Artificial
  Description of Artificial Sequence; Note = Synthetic Construct
cuuauguuguuaugaaauc

SEQ ID NO: 35 - RNA, 19 nt, Artificial
  Description of Artificial Sequence; Note = Synthetic Construct
gccgaagggcaugagcugc

SEQ ID NO: 36 - RNA, 19 nt, Artificial
  Description of Artificial Sequence; Note = Synthetic Construct
gacacucagcccuuggcug

SEQ ID NO: 37 - RNA, 19 nt, Artificial
  Description of Artificial Sequence; Note = Synthetic Construct
gguggcugccuuucugggc

SEQ ID NO: 38 - RNA, 19 nt, Artificial
  Description of Artificial Sequence; Note = Synthetic Construct
ggcccagauaggcacccag

SEQ ID NO: 39 - RNA, 19 nt, Artificial
  Description of Artificial Sequence; Note = Synthetic Construct
caaacggagcugcacuuug

SEQ ID NO: 40 - RNA, 19 nt, Artificial
  Description of Artificial Sequence; Note = Synthetic Construct
caaaugcagacuccuguau

SEQ ID NO: 41 - RNA, 19 nt, Artificial
  Description of Artificial Sequence; Note = Synthetic Construct
cuguuaaccagucagguua

SEQ ID NO: 42 - RNA, 19 nt, Artificial
  Description of Artificial Sequence; Note = Synthetic Construct
uaacacugacugcaugaac

SEQ ID NO: 43 - RNA, 19 nt, Artificial
  Description of Artificial Sequence; Note = Synthetic Construct
cuguuaaccagucagguua

SEQ ID NO: 44 - RNA, 19 nt, Artificial
  Description of Artificial Sequence; Note = Synthetic Construct
uaacacugacugcaugaac

SEQ ID NO: 45 - RNA, 19 nt, Artificial
  Description of Artificial Sequence; Note = Synthetic Construct
ucaggccucagacucccag

SEQ ID NO: 46 - RNA, 19 nt, Artificial
  Description of Artificial Sequence; Note = Synthetic Construct
cucgguguccgaggaccug

SEQ ID NO: 47 - RNA, 19 nt, Artificial
  Description of Artificial Sequence; Note = Synthetic Construct
auaggugauugcucaggac

SEQ ID NO: 48 - RNA, 19 nt, Artificial
  Description of Artificial Sequence; Note = Synthetic Construct
guuccuugagccauucacc

SEQ ID NO: 49 - RNA, 19 nt, Artificial
  Description of Artificial Sequence; Note = Synthetic Construct
cagggcacgcacaccaggc

SEQ ID NO: 50 - RNA, 19 nt, Artificial
  Description of Artificial Sequence; Note = Synthetic Construct
gccagguguggcguuccgu

SEQ ID NO: 51 - RNA, 19 nt, Artificial
  Description of Artificial Sequence; Note = Synthetic Construct
acaggagcuccaggaggaa

SEQ ID NO: 52 - RNA, 19 nt, Artificial
  Description of Artificial Sequence; Note = Synthetic Construct
ucuccucauggagcaccag

SEQ ID NO: 53 - RNA, 19 nt, Artificial
  Description of Artificial Sequence; Note = Synthetic Construct
uguugugaaagcauuauca

SEQ ID NO: 54 - RNA, 19 nt, Artificial
  Description of Artificial Sequence; Note = Synthetic Construct
ugaucaugcauuugaaaca

SEQ ID NO: 55 - RNA, 19 nt, Artificial
  Description of Artificial Sequence; Note = Synthetic Construct
ccaaagagguggcuucuuc

SEQ ID NO: 56 - RNA, 19 nt, Artificial
  Description of Artificial Sequence; Note = Synthetic Construct
gccaagaaggccaucucuu

SEQ ID NO: 57 - RNA, 19 nt, Artificial
  Description of Artificial Sequence; Note = Synthetic Construct
aucuugagauggagagauu

SEQ ID NO: 58 - RNA, 19 nt, Artificial
  Description of Artificial Sequence; Note = Synthetic Construct
aaucuuucagucucaagac

SEQ ID NO: 59 - RNA, 19 nt, Artificial
  Description of Artificial Sequence; Note = Synthetic Construct
gaauagaugacucaaggca

SEQ ID NO: 60 - RNA, 19 nt, Artificial
  Description of Artificial Sequence; Note = Synthetic Construct
ugguuugagucaucuauuu

SEQ ID NO: 61 - RNA, 19 nt, Artificial
  Description of Artificial Sequence; Note = Synthetic Construct
caaguccucuucagaaaug

SEQ ID NO: 62 - RNA, 19 nt, Artificial
  Description of Artificial Sequence; Note = Synthetic Construct
cauuucuuaagaccacuuu

SEQ ID NO: 63 - DNA, 25 bp, Artificial
  Description of Artificial Sequence; Note = Synthetic Construct
caaacaagucagucuaacgaaauta

SEQ ID NO: 64 - RNA, 27 nt, Artificial
  Description of Artificial Sequence; Note = Synthetic Construct
uaauuuccuuagaauggcuuguauuuc

SEQ ID NO: 65 - DNA, 25 bp, Artificial
  Description of Artificial Sequence; Note = Synthetic Construct
auuucucccacgcugacuaccuctc

SEQ ID NO: 66 - RNA, 27 nt, Artificial
  Description of Artificial Sequence; Note = Synthetic Construct
gagagguaagugagcuguggagagaau

SEQ ID NO: 67 - DNA, 25 bp, Artificial
  Description of Artificial Sequence; Note = Synthetic Construct
uuuaguggaauaguugaaaucuatt

SEQ ID NO: 68 - RNA, 27 nt, Artificial
  Description of Artificial Sequence; Note = Synthetic Construct
aauagaauuucaacugacucccuaauu

SEQ ID NO: 69 - DNA, 25 bp, Artificial
  Description of Artificial Sequence; Note = Synthetic Construct
caauucucuguaucuugcauuaaat

SEQ ID NO: 70 - RNA, 27 nt, Artificial
  Description of Artificial Sequence; Note = Synthetic Construct
auuuaauggcaauauuuacagagaaac

SEQ ID NO: 71 - DNA, 25 bp, Artificial
  Description of Artificial Sequence; Note = Synthetic Construct
guuucaugacuuaguguuuaccaaa

SEQ ID NO: 72 - RNA, 27 nt, Artificial
  Description of Artificial Sequence; Note = Synthetic Construct
uuugguauaacacugacugcaugaacc

SEQ ID NO: 73 - DNA, 25 bp, Artificial
  Description of Artificial Sequence; Note = Synthetic Construct
guuucaugacuuaguguuuaccaaa

SEQ ID NO: 74 - RNA, 27 nt, Artificial
  Description of Artificial Sequence; Note = Synthetic Construct
uuugguauaacacugacugcaugaacc

SEQ ID NO: 75 - DNA, 25 bp, Artificial
  Description of Artificial Sequence; Note = Synthetic Construct
ucaaacagacaugauauagcaactg

SEQ ID NO: 76 - RNA, 27 nt, Artificial
  Description of Artificial Sequence; Note = Synthetic Construct
caguugcauaucaacgucuguugaugg

SEQ ID NO: 77 - DNA, 25 bp, Artificial
  Description of Artificial Sequence; Note = Synthetic Construct
ucaaacagacaugauauagcaactg

SEQ ID NO: 78 - RNA, 27 nt, Artificial
  Description of Artificial Sequence; Note = Synthetic Construct
caguugcauaucaacgucuguugaugg

SEQ ID NO: 79 - DNA, 25 bp, Artificial
  Description of Artificial Sequence; Note = Synthetic Construct
aaauucacugcuuuguggcucuuga

SEQ ID NO: 80 - RNA, 27 nt, Artificial
  Description of Artificial Sequence; Note = Synthetic Construct
ucaagagacagcaaacagcagugaaga

SEQ ID NO: 81 - DNA, 25 bp, Artificial
  Description of Artificial Sequence; Note = Synthetic Construct
guuucuuacucagacagaggaggat

SEQ ID NO: 82 - RNA, 27 nt, Artificial
  Description of Artificial Sequence; Note = Synthetic Construct
auccuccucauuucuguaguaagacuu

SEQ ID NO: 83 - DNA, 25 bp, Artificial
  Description of Artificial Sequence; Note = Synthetic Construct
guuucuuacucagacagaggaggat

SEQ ID NO: 84 - RNA, 27 nt, Artificial
  Description of Artificial Sequence; Note = Synthetic Construct
auccuccucauuucuguaguaagacuu

SEQ ID NO: 85 - DNA, 25 bp, Artificial
  Description of Artificial Sequence; Note = Synthetic Construct
cuuuagaugauaagcauuuagccat

SEQ ID NO: 86 - RNA, 27 nt, Artificial
  Description of Artificial Sequence; Note = Synthetic Construct
auggcuaaaucuuucagucucaagacu

SEQ ID NO: 87 - DNA, 25 bp, Artificial
  Description of Artificial Sequence; Note = Synthetic Construct
ccuguuacacagaauaaauccuagt

SEQ ID NO: 88 - RNA, 27 nt, Artificial
  Description of Artificial Sequence; Note = Synthetic Construct
acuaggauugaaauucuguguaacugc

SEQ ID NO: 89 - DNA, 25 bp, Artificial
  Description of Artificial Sequence; Note = Synthetic Construct
augaaaucaaacagacaugaagagg

SEQ ID NO: 90 - RNA, 27 nt, Artificial
  Description of Artificial Sequence; Note = Synthetic Construct
ccucuucaagugcugucugauuccaau

SEQ ID NO: 91 - DNA, 25 bp, Artificial
  Description of Artificial Sequence; Note = Synthetic Construct
aggccucggccaaacacuccgcaag

SEQ ID NO: 92 - RNA, 27 nt, Artificial
  Description of Artificial Sequence; Note = Synthetic Construct
cuugcggaguauuugugcagcgaggga

SEQ ID NO: 93 - DNA, 25 bp, Artificial
  Description of Artificial Sequence; Note = Synthetic Construct
aagccauguuuccuguaauauugcc

SEQ ID NO: 94 - RNA, 27 nt, Artificial
  Description of Artificial Sequence; Note = Synthetic Construct
ggcaauauuuacagagaaacauugugu

SEQ ID NO: 95 - DNA, 25 bp, Artificial
  Description of Artificial Sequence; Note = Synthetic Construct
cugauuucaugacagcaagauaatg

SEQ ID NO: 96 - RNA, 27 nt, Artificial
  Description of Artificial Sequence; Note = Synthetic Construct
cauuaucuuauguuguuaugaaaucaa

SEQ ID NO: 97 - DNA, 25 bp, Artificial
  Description of Artificial Sequence; Note = Synthetic Construct
gccgaagggcaugagcugccagcac

SEQ ID NO: 98 - RNA, 27 nt, Artificial
  Description of Artificial Sequence; Note = Synthetic Construct
gugcuggacacucagcccuuggcugga

SEQ ID NO: 99 - DNA, 25 bp, Artificial
  Description of Artificial Sequence; Note = Synthetic Construct
gguggcugccuuucugggcacaagt

SEQ ID NO: 100 - RNA, 27 nt, Artificial
  Description of Artificial Sequence; Note = Synthetic Construct
acuuguggcccagauaggcacccaggg

SEQ ID NO: 101 - DNA, 25 bp, Artificial
  Description of Artificial Sequence; Note = Synthetic Construct
caaacggagcugcacuuugcacutt

SEQ ID NO: 102 - RNA, 27 nt, Artificial
  Description of Artificial Sequence; Note = Synthetic Construct
aaagugcaaaugcagacuccuguauaa

SEQ ID NO: 103 - DNA, 25 bp, Artificial
  Description of Artificial Sequence; Note = Synthetic Construct
cuguuaaccagucagguuauaccaa

SEQ ID NO: 104 - RNA, 27 nt, Artificial
  Description of Artificial Sequence; Note = Synthetic Construct
uugguauaacacugacugcaugaaccc

SEQ ID NO: 105 - DNA, 25 bp, Artificial
  Description of Artificial Sequence; Note = Synthetic Construct
cuguuaaccagucagguuauaccaa

SEQ ID NO: 106 - RNA, 27 nt, Artificial
  Description of Artificial Sequence; Note = Synthetic Construct
uugguauaacacugacugcaugaaccc

SEQ ID NO: 107 - DNA, 25 bp, Artificial
  Description of Artificial Sequence; Note = Synthetic Construct
ucaggccucagacucccaggagaat

SEQ ID NO: 108 - RNA, 27 nt, Artificial
  Description of Artificial Sequence; Note = Synthetic Construct
auucuccucgguguccgaggaccuggg

SEQ ID NO: 109 - DNA, 25 bp, Artificial
  Description of Artificial Sequence; Note = Synthetic Construct
auaggugauugcucaggacucucct

SEQ ID NO: 110 - RNA, 27 nt, Artificial
  Description of Artificial Sequence; Note = Synthetic Construct
aggagaguuccuugagccauucaccug

SEQ ID NO: 111 - DNA, 25 bp, Artificial
  Description of Artificial Sequence; Note = Synthetic Construct
cagggcacgcacaccaggcuaagac

SEQ ID NO: 112 - RNA, 27 nt, Artificial
  Description of Artificial Sequence; Note = Synthetic Construct
gucuuagccagguguggcguuccguag

SEQ ID NO: 113 - DNA, 25 bp, Artificial
  Description of Artificial Sequence; Note = Synthetic Construct
acaggagcuccaggaggaacaccgc

SEQ ID NO: 114 - RNA, 27 nt, Artificial
  Description of Artificial Sequence; Note = Synthetic Construct
gcggugucuccucauggagcaccaggg

SEQ ID NO: 115 - DNA, 25 bp, Artificial
  Description of Artificial Sequence; Note = Synthetic Construct
uguugugaaagcauuaucaaaugca

SEQ ID NO: 116 - RNA, 27 nt, Artificial
  Description of Artificial Sequence; Note = Synthetic Construct
ugcauuugaucaugcauuugaaacaag

SEQ ID NO: 117 - DNA, 25 bp, Artificial
  Description of Artificial Sequence; Note = Synthetic Construct
ccaaagagguggcuucuucugccac

SEQ ID NO: 118 - RNA, 27 nt, Artificial
  Description of Artificial Sequence; Note = Synthetic Construct
guggcagccaagaaggccaucucuuac

SEQ ID NO: 119 - DNA, 25 bp, Artificial
  Description of Artificial Sequence; Note = Synthetic Construct
aucuugagauggagagauuuagcca

SEQ ID NO: 120 - RNA, 27 nt, Artificial
  Description of Artificial Sequence; Note = Synthetic Construct
uggcuaaaucuuucagucucaagacuc

SEQ ID NO: 121 - DNA, 25 bp, Artificial
  Description of Artificial Sequence; Note = Synthetic Construct
gaauagaugacucaaggcauuuatt

SEQ ID NO: 122 - RNA, 27 nt, Artificial
  Description of Artificial Sequence; Note = Synthetic Construct
aauaaaugguuugagucaucuauuuug

SEQ ID NO: 123 - DNA, 25 bp, Artificial
  Description of Artificial Sequence; Note = Synthetic Construct
caaguccucuucagaaauguauaaa

SEQ ID NO: 124 - RNA, 27 nt, Artificial
  Description of Artificial Sequence; Note = Synthetic Construct
uuuauacauuucuuaagaccacuuucu

(19) United States
(12) Patent Application Publication
(10) Pub. No.: US 2012/0065246 A1
(43) Pub. Date: Mar. 15, 2012

(54) RNA MOLECULES AND THERAPEUTIC USES THEREOF

(75) Inventor: Pal Saetrom, Trondheim (NO)

(73) Assignee: MINA THERAPEUTICS LIMITED, London, GB (GB)

(21) Appl. No.: 13/254,430

(22) PCT Filed: Mar. 1, 2010
(86) PCT No.: PCT/GB2010/000360

§ 371 Date: Nov. 11, 2011

(30) Foreign Application Priority Data
Mar. 2, 2009 (GB) 0903562.7

(51) Int. Cl. A61K 31/713; G06G 7/60; A61P 35/00; C07H 21/02; C07H 1/00

(52) U.S. Cl. 514/44 A; 536/24.5; 536/25.3; 703/11

(57) ABSTRACT

The invention relates to double-stranded RNA molecules in which each strand of said molecule possesses: (a) sufficient complementarity to a target mRNA molecule to facilitate cleavage thereof; and (b) sufficient complementarity to the other strand of the double-stranded RNA molecule so as to form a stable duplex; and in which at least one strand of said molecule possesses: (c) a seed region of complementarity to at least one seed site present in a 3′ untranslated region of at least one target mRNA molecule. The invention also relates to an algorithm for the design of a double-stranded RNA molecule in which each strand of said molecule possesses: (a) sufficient complementarity to a target mRNA molecule to facilitate cleavage thereof; and (b) sufficient complementarity to the other strand of the double-stranded RNA molecule so as to form a stable duplex; and in which at least one strand of said molecule possesses: (c) a seed region of complementarity to at least one seed site present in a 3′ untranslated region of at least one target mRNA molecule; wherein said algorithm comprises the steps: (i) input a population of mRNA sequences transcribed from one or more genes of interest; (ii) identify all subsequences of at least 12 nucleotides in length within the population of step (i) which are complementary to another subsequence of at least 12 nucleotides in length in the population; (iii) determine a list of candidate bi-functional double-stranded RNA molecules, said list comprising the double-strand RNA duplexes comprising the two complementary subsequences of step (ii); and (iv) sort the list of candidate double-stranded RNA molecules of step (iii) based on their potential to cause translational suppression.

The present invention relates to double stranded ribonucleic acid (RNA) molecules (dsRNAs) capable of modulating the expression of target genes and to their design, synthesis and therapeutic uses thereof, particularly in the field of prostate cancer therapy.

RNA interference (RNAi) is a cellular process which results in the down regulation of the expression of target genes. RNAi is mediated by “interfering RNA” (iRNA); an umbrella term which encompasses a variety of double stranded RNA (dsRNA) molecules which function in the RNAi process.

Exogenous dsRNA can be processed by the ribonuclease protein Dicer into double-stranded fragments of 19 to 25 base pairs with several unpaired bases on each 3′ end forming a 3′ overhang. These short double-stranded fragments are termed small interfering RNAs (siRNAs) and these molecules effect the down regulation of the expression of target genes.

siRNAs which function in RNAi contain one strand with a sequence of perfect or near perfect complementarity to a region of a target mRNA transcribed from a target gene. A protein complex known as the RNA-induced silencing complex (RISC), incorporates this strand of the siRNA duplex (the guide strand) and uses it as a template to recognize the target mRNA. RISC is then involved in the cleavage of the target mRNA with perfect or near-perfect complementarity to the incorporated strand and as a result of the cleavage the mRNA can no longer be translated into protein.

Since the elucidation of their function, siRNAs have been used as tools to down-regulate specific genes. They can give transient suppression or, when stably integrated as short hairpins RNAs (shRNAs), stable suppression. siRNAs and shRNAs have been used widely in “knockdown” or “loss of function” experiments, in which the function of a gene of interest is studied by observing the effects of the decrease in expression of the gene. RNAi is considered to have potential benefits as a technique for genomic mapping and annotation. Attempts have also been made to exploit RNA interference in therapy e.g. in the inhibition of viral gene expression and in the knockdown of host receptors and co-receptors for viruses. It is speculated that siRNA and related molecules could be used in treatments for a wide variety of diseases including neurodegenerative diseases and particularly cancer, for example by silencing genes which are up-regulated in tumor cells or genes involved in the progression of the cell cycle.

There are, however, numerous difficulties associated with the design of effective siRNA molecules. While any siRNA sequence of 19-25 nucleotides in length with perfect or near perfect complementarity to an mRNA target sequence can target that sequence, the effectiveness of gene silencing is not identical in all cases. Numerous factors including the degree of mismatching, nucleotide repeats, GC-content and thermodynamic end stability, contribute to the effectiveness of the molecule.

A further problem is that dsRNA molecules induce a number of undesirable cellular responses, most notably the production of pro-inflammatory cytokines and the induction of the interferon response, which can lead to undesired global translational arrest. In addition, it is believed that RNAi machinery can become saturated, leading to the accumulation of toxic iRNA processing precursors. Such responses and toxicity can be limited by using a low concentration of siRNA. Therefore it is desirable to design siRNA molecules which are efficient as well as effective so that a lower concentration can be used to achieve the'desired result.

A significant further difficulty with siRNA techniques are so-called “off-target effects”. These are caused when the siRNA guide strand incorporated into the RISC complex has complementarity not only to the target mRNA but also to one or more additional mRNA sequences. As a result of this coincidental complementarity, unintentional silencing of one or more non-targeted genes occurs. The problem of off-target effects is compounded by the fact that the siRNA passenger strand, i.e. the strand of identical or near identical sequence to the mRNA target sequence, can be incorporated into RISC instead of the intended guide strand. This not only reduces the efficiency of the siRNA method since the target mRNA is not effectively targeted, but it increases the likelihood of further off-target effects against other non-target genes. It is estimated that the error rate of off-target interactions with standard siRNA molecules is about 10%. Off-target effects reduce the efficiency of siRNA studies and have potentially dangerous consequences in medical applications.

Off target effects can be limited by the use of a low concentration of siRNA and so there exists a need for methods of designing efficient siRNA molecules, i.e. those with maximum gene-silencing activity and minimum off-target effects and which therefore are required in a lower concentration to achieve the desired function. Numerous computational tools have therefore been developed in attempts to identify likely successful siRNA molecules that maximize the extent of gene knockdown but minimize off-target effects.

Many previous attempts have stemmed from the observation that dsRNA molecules termed Dicer-substrate siRNAs are more potent modulators of gene expression than standard siRNA molecules. Dicer-substrate siRNAs are longer than standard siRNAs and, as the name suggests, resemble Dicer substrates. As mentioned above, Dicer cleaves dsRNA into siRNA, however, it also performs the function of loading the siRNA molecule into the RISC complex. This explains why siRNAs which are produced from Dicer-substrate siRNAs are more efficient at gene silencing than siRNAs introduced directly into the cell. While the use of Dicer-substrate siRNAs has benefits over the use of standard siRNAs, it does not overcome the off-target effects of the guide strand or the incorrect loading of the passenger strand into the RISC complex entirely.

Methods which have sought to reduce off-target effects of the passenger strand have been focused primarily on preparing siRNAs in which the passenger strand, as well as the guide strand, has complementarity to a target mRNA sequence. In Hossbach et al., (2006) RNA Biology 3 (2): 82-89 a method of siRNA design is disclosed in which both strands of the resulting siRNA molecule are “guide strands” i.e. both strands are complementary to at least one target mRNA sequence and are sufficiently complementary to each other such that a stable siRNA duplex can be formed with the characteristic 3′ overhanging ends of siRNA molecules.

Despite the advances made in improving the effectiveness of siRNAs, there remains a need for dsRNA molecules which possess further increased efficiency and further reduced off-target effects and toxicity. Methods of designing such improved dsRNA molecules are therefore also desirable. dsRNA molecules with improved efficiency and reduced off-target effects would have potential benefits in a variety of applications, in particular in the study of gene function and in therapeutic methods.

Micro-RNAs (miRNAs) are a type of dsRNA molecule that also function in the modulation of gene expression. Like siRNAs, miRNAs are non-coding dsRNAs, however, unlike siRNAs, they have been identified as endogenous dsRNA molecules. Once processed into mature dsRNA molecules, miRNA molecules are nevertheless structurally similar to siRNAs produced from exogenous dsRNA; siRNAs (and shRNAs) resemble intermediates in the processing pathway of the endogenous miRNA genes.

miRNAs, like siRNAs, use RISC to down-regulate target genes, but unlike siRNAs, most animal miRNAs do not cleave the target mRNA molecule. Instead, animal miRNAs preferentially target sites with imperfect complementarity in the 3′ untranslated regions (UTRs) of the target mRNA sequence and reduce protein output through a combination of translational suppression and subsequent polyA removal and mRNA degradation.

miRNA binding sites within a target mRNA are usually located within the mRNA 3′ untranslated region (3′ UTR). In contrast to cleavage, translational suppression only requires base-pairing between the mRNA target and nucleotides 2 to 8 from the 5′ end of the dsRNA's guide strand. This region of the siRNA strand, known as the seed region, is critical for miRNA targeting and although mRNA target seed sites with imperfect seed-pairing to the seed region can be responsive, the majority of miRNA seed regions have perfect seed pairing with the target mRNA seed site. Because siRNAs and miRNAs are interchangeable, exogenous siRNAs will down-regulate mRNAs with seed complementarity to the siRNA.

Perfect seed complementarity does not guarantee down regulation, however. Instead, multiple factors that characterize a seed site's sequence context determine the regulatory potential of each site. More important than the characteristics of individual sites, however, is the number of seed sites within a 3′ UTR. Multiple target sites within a 3′ UTR give synergistic down regulation, but only if the distance between the start of the target sites is in an optimal range of about 14 to 46 nucleotides. Moreover, different dsRNAs can also cooperate and give synergistic down regulation as long as their sites are located within this optimal range. This synergistic regulation means that pairs of target sites located within an optimal distance range have a much higher regulatory potential than individual isolated sites.

As is customary in the art and for convenience, “siRNA” is used herein to refer to RNA molecules which function through cleavage of target mRNA molecules, induced by base-pair binding to a coding region of an mRNA molecule. “miRNA” is used to refer to RNA molecules which through base-pair binding to short (e.g. 7 nucleotides) seed sequences within the 3′UTR of an mRNA molecule inhibit normal utilization of mRNA, thereby down regulating gene expression without the need to induce cleavage of target molecules. The terms are also used to refer to those different activities and modes of action.

WO 2008/094516 describes molecules which utilize siRNA and miRNA technology in a single molecule. Examples 1 and 2 therein describe sense strands with siRNA activity against genes involved in HIV infection which also incorporate miRNA-like sites for a target HIV 3′UTR. Following the Hossbach et al. (supra) principles Example 3 therein describes an siRNA duplex in which each strand has siRNA cleavage functionality (one strand per target gene). As a variation, the final Example, Example 4, describes a duplex RNA molecule in which the top strand functions as miRNA and the bottom strand functions as an siRNA. Thus duplexes are described with 2-way functionality against a common or different target genes.

The present inventor has gone beyond the multi-functioning RNA molecules of the prior art to generate dsRNA molecules with at least 3-way, preferably 4-way functionality, each strand down-regulating the expression of one or more target genes by cleavage of target messenger RNA (siRNA activity) and at least one strand, preferably both strands also down-regulating the expression of one or more target genes by translational suppression of the mRNA (miRNA activity).

The present invention provides an algorithm for the design of multi-targeting dsRNA molecules which have increased efficiency and reduced off-target effects. In common with the dsRNA molecules previously described, for instance in Hossbach et al., RNA Biology 3 (2): 82-89, both strands of the dsRNA molecules designed by the algorithm of the present invention are complementary to at least one mRNA target sequence and function in modulation of gene expression by mediating cleavage of the target mRNA sequence(s).

Furthermore, the algorithm of the present invention results in the design of dsRNAs in which at least one strand, preferably both strands of the dsRNAs also target specific mRNA target transcripts for 3′ UTR-mediated translational suppression. In other words, siRNA-like and miRNA-like targeting has been exploited and the novel design algorithm of the present invention results in the design of dsRNAs in which at least one strand, preferably both strands target(s) both types of target sites in an mRNA molecule, with the effect that both strands function in the down regulation of gene expression by cleavage and at least one strand, preferably both strands function in the downregulation of gene expression by 3′UTR-mediated translational suppression.

The algorithm of the present invention results in the design of dsRNA molecules which have increased gene-silencing efficiency since there are at least three, preferably four potential regulatory activities possessed by each dsRNA duplex. The dsRNA molecules of the present invention are particularly advantageous when it is desired to target two or more genes for silencing simultaneously. In diseases, for example cancers such as prostate cancer where multiple genes are implicated, such a multi-targeting approach is highly desirable.

The dsRNA molecules of the present invention have an increased gene-silencing efficacy compared to those of the prior art. They are more effective due to their increased number of activities and can be designed to have further advantageous properties such as reduced degrees of mismatching to target sequences, reduced nucleotide repeat sequences, optimal GC percentages, reduced probabilities of cleavage-based off target effects and lower thermodynamic end stability.

The increased efficacy of the dsRNAs of the present invention allows them to be used in a lower concentration than the dsRNAs of the prior art in order to achieve the desired effect. The dsRNAs of the present invention therefore result in a reduction of the problems associated with using high levels of siRNA such as the induction of pro-inflammatory cytokine production and of innate immune response such as the interferon response. A lower degree of saturation of the iRNA machinery can be achieved, which avoids subsequent accumulation of toxic iRNA processing precursors within the cell.

In one aspect, the present invention provides an algorithm for the design of a double-stranded RNA molecule in which each strand of said molecule possesses:

a) sufficient complementarity to a target mRNA molecule to facilitate cleavage thereof; and

b) sufficient complementarity to the other strand of the double-stranded RNA molecule so as to form a stable duplex;

and in which at least one strand of said molecule possesses:

c) a seed region of complementarity to at least one seed site present in a 3′ untranslated region of at least one target mRNA molecule.

Preferably the subsequences are at least 12 nucleotides in length and said algorithm comprises the following steps:

(i) Input a population of mRNA sequences transcribed from one or more genes of interest;

(ii) Identify all subsequences of at least 12 nucleotides in length within the population of step (i) which are complementary to another subsequence of at least 12 nucleotides in length in the population;

(iii) Determine a list of candidate bi-functional dsRNAs, said list comprising the double-strand RNA duplexes comprising the two complementary subsequences of step (ii); and

(iv) Sort the list of candidate dsRNAs of step (iii) based on their potential to cause translational suppression.

More preferably the subsequences are 15 to 22 nucleotides in length and said algorithm comprises the following steps:

(i) Input a population of mRNA sequences transcribed from one or more genes of interest;

(ii) Identify all subsequences of 15 to 22 nucleotides in length within the population of step (i) which are complementary to another subsequence of 15 to 22 nucleotides in length in the population;

(iii) Determine a list of candidate bi-functional dsRNAs, said list comprising the double-strand RNA duplexes comprising the two complementary subsequences of step (ii); and

(iv) Sort the list of candidate dsRNAs of step (iii) based on their potential to cause translational suppression.

Preferably the subsequences identified in step (ii) of the above method are the same length as the subsequences to which they are complementary. Preferably the subsequences are 19mers which are complementary to another 19mer in the population. As a result, the dsRNAs in steps (iii) and (iv) are 19mer dsRNAs. If the required threshold on complementarity is maintained, then using shorter subsequences may result in the identification of a greater number of duplex candidates which may be less stable. In contrast, if the required threshold on complementarity is maintained, then using longer subsequences may result in the identification of fewer duplex candidates which may be more stable. The skilled man will be able to adjust these parameters as required given the particular aim of the process and the size and nature of the population of mRNA sequences in step (i).

For convenience, and as a preferred embodiment, the term “19mer” will be used to refer to the subsequences identified at step (ii) above and the corresponding duplexes of step (iii). However, unless otherwise clear from the context, it will be appreciated, as discussed above, that somewhat longer or shorter sequences may be employed.

As used herein, the term “RNA” means a molecule comprising at least one ribonucleotide residue. By “ribonucleotide” is meant a nucleotide with a hydroxyl group at the 2′ position of a beta-D-ribo-furanose moiety. The terms include double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the present invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “double stranded RNA” or “dsRNA” as used herein refers to a ribonucleic acid duplex, including but not limited to, endogenous and artificial siRNAs, short hairpin RNAs (shRNAs) and miRNAs.

The term “short interfering RNA” or “siRNA” as used herein refers to a nucleic acid molecule capable of modulating, by inhibiting or down regulating, gene expression, through RNAi or gene silencing via sequence-specific-mediated cleavage of one or more target mRNA strands.

The term “microRNA” or “miRNA” refers to a nucleic acid molecule capable of modulating, by inhibiting or down-regulating, gene expression through sequence-specific-mediated translational suppression and subsequent polyA removal and degradation of one or more target mRNA strands.

Typically, siRNA functions by mediating the cleavage of mRNA target sequences which possess a region of complete or near complete complementarity to the “guide strand” of the siRNA molecule. Typically miRNA functions by mediating translational suppression and subsequent polyA removal and degradation of mRNA target sequences which possess seed sites within their 3′ untranslated regions (3′ UTR) which are complementary to nucleotides 2 to 8 from the 5′ end of the miRNA's guide strand (the seed region). However, because siRNA and miRNA molecules are structurally related, siRNAs can function as miRNAs and vice versa.

By “complementarity” and “complementary” are meant that a nucleic acid can form hydrogen bond(s) with another nucleic acid for example by Watson-Crick base pairing. A nucleic acid which can form hydrogen bond(s) with another nucleic acid through non-Watson-Crick base pairing also falls within the definition of having complementarity. A percent complementarity indicates the percentage of residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

In the context of the present invention, siRNA-cleavage activity requires a high degree of complementarity, thus a guide strand will have no more than 5, preferably no more than 4 or 3, most preferably 2, 1 or no mismatches with a region of a target mRNA molecule. In the context of complementarity between seed regions and seed sites, the seed region will have no more than 3, preferably no more than 2 or 1, most preferably no mismatches with the target mRNAs' seed site(s).

“Perfectly complementary” or “fully complementary” means that all sequential residues of a nucleic acid sequence will form hydrogen bonds with the same number of sequential residues in a second nucleic acid sequence.

A “bifunctional” dsRNA molecule is a dsRNA molecule with two known and desired iRNA targeting functions. Typically, this term refers to a dsRNA molecule in which both strands have complementarity to one or more target mRNA sequences and thereby target the mRNA sequences for cleavage (siRNA activity).

A “target gene” or “gene of interest” is a gene whose expression is desired to be modulated. The term includes any nucleotide sequence, which may or may not contain identified gene(s), including, but not limited to, coding region(s), non-coding region(s), untranscribed region(s), intron(s), exon(s) and transgenes(s). The target gene can be a gene derived from a cell, an endogenous gene, a transgene or exogenous genes such as genes of a pathogen, which is present in the cell after infection thereof. The cell containing the target gene can be derived from or contained in any organism. A “target mRNA” sequence is an mRNA sequence derived from a target gene.

A “19mer subsequence” is a sequential section of gene, mRNA or RNA sequence which is 19 nucleotides in length.

By “seed site” is meant a nucleotide sequence present in the 3′ UTR of a target mRNA sequence which is complementary to the seed region of at least one strand of a dsRNA molecule and which has the potential to mediate miRNA-like translational suppression and/or polyA removal and subsequent degradation of the mRNA strand it is contained within when hybridised to its complementary seed region.

By “seed region” is meant a nucleotide sequence present on a strand of the dsRNA molecules described herein which is complementary to one or more seed sites present in the 3′ UTR of one or more target mRNA molecules. Typically, the seed region comprises nucleotides 2 to 8 from the 5′ end of the dsRNA strand, i.e. 7 nucleotides in length, however the seed region may be 6 to 10 residues in length. Seed regions will preferably start at nucleotide 2 from the 5′ end. Longer seed regions are likely to result in stronger miRNA-like down-regulation but are likely to reduce the number of candidate dsRNAs identified by the algorithm of the present invention. In contrast, shorter seed regions are likely to result in weaker miRNA-like down-regulation but are likely to increase the number of candidate dsRNAs identified by the algorithm of the present invention. The skilled man will be able to decide what length of seed region to use. Preferably the residue at position 1 from the 5′ end of the dsRNA strand is adenosine. When both strands of a dsRNA molecule contain seed regions, as is preferred, the seed regions of the two strands are optionally, but not necessarily, non-identical to each other.

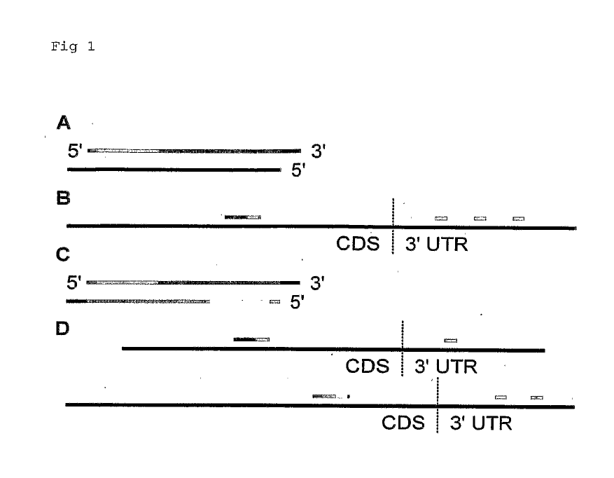

A given seed region may be complementary to seed sites in more than one target mRNA molecule (see FIG. 1). The seed sites may be within the 3′UTR of the same mRNA molecule which is targeted for cleavage by the strand incorporating a corresponding seed region, thus the two activities of the strand work in cis or the seed sites may be within the 3′UTR of a different mRNA molecule, i.e. the activities work in trans. FIG. 1(D) shows how each strand is working both in cis and in trans.

The algorithm of the present invention designs dsRNA molecules which are more effective and efficient modulators of gene expression than those designed by previously described algorithms. By “modulate” is meant that the up-regulation or down-regulation of the expression level of a gene(s), or the levels of the polypeptide(s) encoded by a gene or the activity thereof, or levels of the RNA molecule(s) transcribed from a gene as compared to the level in the absence of the dsRNA molecules designed by the algorithm of the present invention.

Thus, in a further aspect the present invention provides a double-stranded RNA molecule in which each strand of said molecule possesses:

a) sufficient complementarity to a target mRNA molecule to facilitate cleavage thereof; and

b) sufficient complementarity to the other strand of the double-stranded RNA molecule so as to form a stable duplex, and in which at least one strand of said molecule possesses:

c) a seed region of complementarity to at least one seed site present in a 3′ untranslated region of at least one target mRNA molecule.

Preferably each strand of the duplex is at least 12, more preferably at least 15, more preferably 17, still more preferably at least 19 nucleotides in length. Preferably the duplex is hybridised over a length of at least 12, more preferably at least 15, more preferably 17, still more preferably at least 19 nucleotides. Each strand may be exactly 19 nucleotides in length or in a preferred embodiment one strand is 25 nucleotides and the other 27 nucleotides in length. Preferably the duplex length is less than 30 nucleotides since duplexes exceeding this length may have an increased risk of inducing the interferon response. The strands forming the dsRNA duplex may be of equal or unequal lengths.

Various additions to the basic algorithm are described herein and the resulting molecules encompass further aspects and embodiments of the present invention.

By “inhibition”, “down-regulation” and “gene silencing” is meant the reduction of the expression level of a gene(s), or levels of the polypeptide(s) encoded by a gene or the activity thereof, or levels of the RNA molecule(s) transcribed from a gene below that observed in the absence of the dsRNA molecules designed by the algorithm of the present invention.

The “guide strand” of a dsRNA molecule is the strand which is incorporated into the RISC protein, the other strand being termed the “passenger strand”. In the dsRNA molecules of the present invention both strands are desired to be incorporated into RISC to illicit down-regulation of a target mRNA sequence and so both strands can be considered to be guide strands.

Preferably, but not essentially, the population of mRNA sequences provided in step (i), i.e. the mRNA target sequences, comprises a plurality of different mRNA sequences. In a preferred embodiment the plurality of mRNA sequences are transcribed from more than one gene of interest. Optionally, more effective down regulation of an intended target gene can be achieved by also targeting other genes, for instance genes which encode transcription factors which positively regulate the expression levels or activity of the intended target gene, RNA molecule or protein.

Optionally, the population comprises a plurality of mRNA sequences which are transcribed from a single target gene, for instance by alternative gene splicing. Optionally, the population consists only of one target mRNA sequence and each strand of the dsRNA molecule designed by the present algorithm has complementarity to a different, optionally overlapping, region of that mRNA molecule. Alternatively, but rarely, the population of mRNA sequences comprises only one mRNA sequence and both strands of the dsRNA molecule designed by the present algorithm have complimentarily to the same, palindromic, region of the mRNA molecule.

In other words the dsRNAs designed by the algorithm of the present invention may target a single or multiple target genes. In one embodiment the dsRNA molecules are designed so that each strand targets separate mRNA targets and preferably separate genes.

Optionally the genes of interest are implicated in diseases. Preferably the disease is cancer, most preferably prostate cancer.

Step (ii) of the above algorithm requires the identification of all 19mer subsequences within the mRNA input population of step (i) which are complementary to at least one other 19mer subsequence in the population. The identification of subsequences with such complementarity can be performed by any method known in the art. Preferably, the method used is that set out in Hossbach et al. (supra). In accordance with this method, the Perl script accessible at http://www.mpibpc.mpg.de/groups/luehrmann/siRNA is used. This program accepts input cDNA or mRNA sequences in raw format and computes the degree of complementarity for all possible base-paired combinations of 19mer subsequences of the input sequences.

To be functional in iRNA, bi-functional dsRNA duplexes must anneal and form stable duplexes under biological conditions, such as the conditions found in the cytoplasm of a cell. The number of base pairs present in a duplex helps to determine the stability of the duplex. Therefore, in step (ii) of the present algorithm, the 19mer subsequences identified within the input population can be selected based on the extent of their complementarity to one or more other 19mer subsequence within the input population.

Preferably, the algorithm identifies only those 19mer dsRNA molecules in which both strands have near perfect, more preferably perfect, complementarity to one or more other 19mer sequence in the population. This results in the design of 19mer dsRNA molecules in which both strands have perfect or near perfect complementarity to the one or more target mRNA molecules.

In a yet further preferred embodiment, the algorithm identifies only those dsRNA molecules in which there are at most 5 base pair mismatches between the two 19mer strands of the duplex, preferred duplexes have at most 4 or 3 base pair mismatches.

In other words, in a preferred embodiment step (ii) is as follows:

(ii) Identify all 19mer sub-sequences within the population of step (i) which are complementary to any other 19mer subsequence in the population, wherein there are at most five base pair mismatches between the two 19mer sub-sequences.

A “base-pair mismatch” is a nucleotide in a nucleic acid sequence comprising one strand of a double-stranded nucleic acid duplex which does not form a Watson-Crick base pair with the nucleotide of the second nucleic acid sequence which is located spatially opposite to it in the duplex structure.

The number of mismatches between the two 19mer subsequences of step (ii) can be determined according to any known method in the art. For instance, the number of mismatches between two aligned sequences can be measured by standard edit distance; that is, the number of mismatches between two sequences is the minimum number of insertions, deletions, or substitutions needed to transform the reverse complement of first sequence to the second sequence.

Given two target mRNA sequences transcribed from one or more genes of interest (input sequences s₁ and s₂) the list of candidate bi-functional siRNAs will consist of all pairs of 19mer subsequences, said subsequences being termed s_{1,i} in s₁ and s_{2,j} in s₂, such that m(s_{i,i}, s_{2,j})≦5, where m(s_{1,i}, s_{2,j}) calculates the number of mismatches when the reverse-complement of s_{1,i} is aligned to s_{2,j.}

Standard edit distance allows for insertions and deletions in addition to substitutions. Consequently, when using standard edit distance to identify dsRNA candidates in step (ii) of the design algorithm, the resulting dsRNAs may become asymmetric; that is, the predicted duplexes may contain asymmetric bulges and internal loops. If desired, as an optional alternative to the standard edit distance method, the number of mismatches between the two 19mer subsequences of step (ii) can be determined using a restricted method in which only substitutions are allowed in the edit distance computation. If it is desired to filter out asymmetric candidates, a Hamming distance measure, which is well-known in the art, can be used; the Hamming distance is the number of single nucleotide mismatches when not allowing insertions or deletions.

Alternatively, a modified version of the Smith-Waterman algorithm (T F Smith & M S Waterman (1981) Journal of molecular biology 147: 195-197) can be used such that for each 19mer subsequence s_{1,i} in s_{2,j}, a search is performed for all 19mer subsequences s_{2,j} in s₂ such that the edit distance between s_{2,j} and the reverse complement of s_{1,i} is at most 5.

When determining the number of base-pair mismatches for the purposes of screening the identified 19mer subsequences on the basis of their degree of complementarity to other 19mer subsequences in the population, the G/U wobble content and the frequency of other non-Watson-Crick base pairs can be considered. Optionally, certain non-Watson-Crick base pairs, for instance G/U base pairs and those disclosed in Leontis et al., (2002) Nucleic Acids Research Vol. 30, No. 16, pp 3497-3531, can be considered not to be true “base-pair mismatches” for the purposes of screening. Thus, if desired, a certain number of one or more particular non-Watson-Crick base-pairs may be accepted in addition to the 5 or fewer base-pair mismatches. Preferably there will be no more than 3, more preferably no more than 2, most preferably 1′ or 0 such base-pairs in addition to the 5 or fewer true mismatches.

The dsRNA molecules designed by the algorithm of the present invention are ideally thermodynamically stable duplexes. In addition, when designing dsRNA molecules for the modulation of the expression of a target gene, it is important to consider the fact that dsRNAs can induce cleavage of non-target transcripts that have a limited number of mismatches to the dsRNA guide strand which is incorporated into the RISC protein complex. This reduces the efficiency of the dsRNA molecule and is therefore not desired. Consequently, dsRNA molecules should have limited complementarity to transcripts other than the intended target to prevent unintended off-target effects.

Therefore, in a preferred embodiment, the method comprises an additional step (iii)(a) following step (iii) or (iv):

From the list of candidate 19mer dsRNAs, remove all dsRNAs that
(p) contain one or more of the motifs aaaa, cccc, gggg, or tttt in either strand; and/or
(q) have a GC-percentage less than 25% or greater than 75% in either strand; and/or
(r) have a high probability of cleavage-based off-target effects; and/or
(s) have a large difference in duplex thermodynamic end stability.

Tools and algorithms for identifying dsRNAs in steps (p) and (q) are well known to the skilled artisan. Such algorithms include those described and references in Chalk et al., (2004) Biochem Biophys Res Commun 319: 264-274, Neale et al., (2005) Nucleic Acids Res 34: D140-144, Saetrom, (2004) Bioinformatics 20: 3055-3063, Saetrom and Shove, (2004) Biochem Biophys Res Commun 321: 247-253 and Vert et al., (2006) BMC Bioinformatics 7: 520 (17 pages).

The absolute value of the difference in duplex thermodynamic end stability (ΔΔG) can be calculated in accordance with any method standard in the art. Optionally, the absolute value of the difference in duplex thermodynamic end stability is calculated by RNAfold (Hofacker et al., (2003) Nucleic Acids Research Vol. 31, No. 13, pp 3429-3431) by considering the 5 closing nucleotides at the ends of the 19mer duplex. Preferably the absolute value of the difference in duplex thermodynamic end stability as calculated by RNAfold is less than 3 kcal/mol, more preferably less than 1 kcal/mol. Thus, preferably step (iii)(a) involves the removal from the list of candidate 19mer dsRNAs, of all dsRNAs that have an absolute value of the difference in duplex thermodynamic end stability which is greater than 3 kcal/mol, preferably which is greater than 1 kcal/mol.

The probability of a dsRNA candidate having cleavage-based off-target effects is a function of its complementarity to non-target mRNA sequences and can be determined by any known method in the art. Optionally, an ungapped Smith-Waterman method (T F Smith & M S Waterman (1981) Journal of molecular biology 147: 195-197) can be used to screen both strands of the candidate dsRNA against the Ensembl (Flicek, P., et al. (2008) Ensembl 2008. Nucleic Acids Res 36: D 707-714) human transcriptome database (Snøve, O., Jr., et al. (2004) Biochem Biophys Res Commun 325: 769-773) to identify a dsRNA's potential off-target transcripts. Alternatively, both strands of the dsRNA candidate can be screened against a population of chosen mRNA sequences, for example a selection of GenBank sequences, which do not encompass the entire Ensembl human transcriptome database. Alternatively a Hamming distance measure can be used.

Preferably, dsRNA molecules are selected if both strands of the molecule have more than two mismatches to the identified off-target transcripts and if less than ten identified transcripts have only three mismatches to the given dsRNA. Conversely, preferably dsRNA molecules are not selected either if one or both strands have two or fewer mismatches to the identified off-target mRNA transcripts and/or if more than ten identified mRNA transcripts have only three mismatches to the given one or both strands of the dsRNA.

Optionally, step (iii)(a) of the algorithm also comprises the step of identifying and selecting dsRNA molecules from the candidate list which have characteristics in common with known highly effective standard siRNAs. This step can be performed using any sequence-based algorithm known in the art. Preferably, this step comprises removing from the list of candidate dsRNAs, those dsRNAs in which one or both strands have a GPboost score of less than 0.1. GPboost is a known genetic programming based prediction system of siRNA efficacy and the methods used for determining the GPboost score of siRNA strands is disclosed in “Predicting the efficacy of short oligonucleotides in antisense and RNAi experiments with boosted genetic programming”, P\dot{a}l Saetrom (2004) Bioinformatics 20(17): 3055-3063, the content of which is incorporated here by reference. Alternatively or in addition, this step comprises the steps of the algorithm described by Reynolds [Reynolds et al. (2004) Nature biotechnology 22(3):326-330], which is incorporated here by reference. These steps are used to filter out siRNA molecules which lack sufficient specific sequence features which are associated with highly effective siRNAs. One of ordinary skill in the art would be able to define and refine his threshold for his particular purpose.

Step (iv) of the algorithm requires the sorting of the list of candidate 19mer dsRNAs based on their potential to cause translational suppression. dsRNAs, including siRNAs, cause miRNA-like translational suppression via the binding of their “seed region” to one or more “seed sites” typically found in the 3′ UTRs of the target mRNA sequence(s). While additional complementarity between the dsRNA strand and the mRNA sequence may exist, only the seed region of the dsRNA strand and the seed site of the mRNA target need to be complementary in order to function.

At least one, preferably both of the strands in the dsRNAs of the present invention function in miRNA-like translational suppression via the binding of their seed region to one or more seed sites in the 3′UTRs of the target mRNA sequences(s). Preferably, the seed region of the strands of the dsRNA molecule which have complementarity to at least one seed site present in a 3′ untranslated region of at least one target mRNA molecule is the region from nucleotides 2 to 8 from the 5′ end of the strand.

Where the duplex targets two (or less usually more than two) mRNA molecules, preferably each target has a functioning seed site. Preferably each strand has a seed region but it is possible that the seed region of one strand targets seed sites in both (or each) target mRNAs and the other strand has no seed region (e.g. PCS16 in our Examples). Such a duplex still has four regulatory functions, i.e. four activities. In less preferred molecules one target mRNA molecule may have no seed sites (e.g. PCS 18 in our Examples). In a yet further embodiment, one target mRNA molecule may have seed sites complementary to the seed regions of each strand of the duplex, while the other target (which is cleaved) has no seed sites; a duplex acting in this way still has four activities.

Optionally, the seed region in at least one, preferably both of the two dsRNA strands has near perfect, more preferably perfect, complementarity to the one or more mRNA target seed sites. A given bifunctional strand may target different target genes for both its siRNA and miRNA like downregulation (trans). Alternatively a bifunctional strand may target the same target gene for both its siRNA and miRNA like downregulation (cis). A strand may operate in both cis and trans as shown in FIG. 1(D) and thus be essentially trifunctional, in a given duplex one or both of the strands may have this trifunctionality.

The presence of multiple seed sites in the mRNA target increases a dsRNA's regulatory potential and multiple optimally spaced seed sites give synergistic regulation.

Optionally, to model a dsRNA's potential for causing translational suppression, a model is used that takes into account the number of seed sites and the distance between these seed sites in the target mRNA sequence(s). Such a model is discussed in WO 2003/094516. In addition, it is optionally assumed that the dsRNA's maximum number of consecutively optimally spaced seed sites mostly determines the dsRNA's regulatory potential, that additional seed sites have a smaller impact on regulatory potential, and that this impact depends on their individual distance.

The preferred model uses a concept called “seed site modules”. Seed site modules are sets of seed sites where the maximum distance between consecutive seed sites is between 13 and 100 nucleotides, preferably 13 to 35 nucleotides, more preferably 17 to 35 nucleotides and most preferably 21 to 25 nucleotides. In the preferred model, to score the regulatory potential of a dsRNA:
(t) identify the mRNAs' seed sites complementary to the seed region of one or both strands of the dsRNA;
(u) structure the seed sites into seed site modules; and
(v) score individual seed site modules to identify the module with the highest regulatory potential.

In the above model, the population of mRNAs in which complementary seed sites are identified can comprise any desired population of mRNAs. In one embodiment the population is the population of mRNA sequences transcribed from one or more genes of interest referred to in step (i) of the algorithm of the present invention. Alternatively the population may comprise additional mRNA sequences derived from additional genes of interest. In a yet further embodiment the population comprises only the two mRNA molecules which are complementary to the two strands of the dsRNA molecule of the invention, i.e. the two mRNA molecules which are targeted by the dsRNA for siRNA-like down regulation by cleavage.

In step (v) above, the score for individual seed site modules is determined as the number of seed sites within the module. Since seed sites that are less than 17 nucleotides apart may have reduced or no cooperative effect, the number of closely spaced seed sites may be subtracted when computing the module score. In this regard “closely spaced seed sites” are those less than 17 nucleotides apart, optionally less than 13 nucleotides apart. The score for the highest scoring seed site module plus a distance-dependent contribution from the other seed site modules then gives the siRNA's regulatory potential.

The above model may be performed as follows: given a sequence of n seed sites S={s₁, . . . , sₙ} and a sequence of n−1 distances D={d₁, . . . dₙ₋₁}, which correspond to the distances between consecutive seed sites such that dᵢ is the distance between seeds s₁ and sᵢ₊₁. A seed site module Mᵢ is then defined as Mᵢ={sᵢ, . . . , sⱼ}, where ∀iε{i, . . . , j−1}dᵢ≦35. In other words, the distance between all consecutive seed sites in a seed site module is at most 35 nucleotides. Consequently, for a set of seed sites S, there is a corresponding set of seed site modules SM={M₁, . . . , Mₖ}, where k≦n−1. In other words, any set of seed sites can be structured into a smaller or equal sized set of seed site modules. The score f for seed site module Mᵢ is f(Mᵢ, D)=1+Σᵢ₌ᵢ=g(dᵢ), where g(dᵢ)={0 if dᵢ≦13; 1 if dᵢ≧17; and (dᵢ−13)/4 otherwise}. To illustrate, a seed site module that consists of four seed sites with distances {10, 30, 15} has a score f=1+g(10)+g(30)+g(15)=1+0+1+ 2/4=2.5. Finally, the score F for a siRNA with seed sites S, distances D, and seed site modules SM is F(S, D, SM)=f(Mₘ, D)+Σ_{Miε{M1, . . . , Mm}}f(Mᵢ, D)*h(dᵢ₋₁)+Σ_{Miε{Mm′, . . . , Mk}}f(Mᵢ, D)*h(dᵢ₋₁), where m=i, max {f(Mᵢ, D), MᵢεSM} (the index of the highest scoring seed module), m′ is the index of the seed module following m, and h(dᵢ₋₁)={if dᵢ₋₁≧70; (70−dₘ)/35 otherwise}. In other words, the score is given by the highest scoring seed site module Mₘ plus the scores of the remaining seed site modules weighted by the distance between the individual seed site modules. To illustrate, an siRNA that has three seed site modules with scores {2, 4, 3} and individual distances {1000, 50} has a score F=4+2*h(1000)+3*h(50)=4+2*+3*2=6.21. Scores calculated in this step are termed “miRNA scores” and reflect the likelihood of the strand to cause miRNA-like translation inhibition.

It is clear that the term “sorting” in the context of the present invention means “ranking in order followed by selection”. In all steps of the present invention which involve sorting, preferred candidate dsRNAs are selected based on the sorted list. Sorting without selection is a mere arrangement of information. It is within the competence of the skilled man to determine his tolerance in a given context and to select the desirable dsRNAs from the sorted list of candidate dsRNAs.

As discussed above, the algorithm of the present invention designs dsRNAs in which each strand possesses siRNA-like functionality and at least one strand, preferably both strands possess(es) mi-RNA like RNAi functionality.

Optionally the length of the strands is selected by changing the parameters of step (i) of the above method so that rather than 19mer subsequences within the target mRNA population being identified, subsequences of different lengths are identified. Alternatively, 19mer subsequences are identified (as is typical), followed by an additional subsequent step in which extensions to the 19mer strands comprising the candidate dsRNA are designed. This additional extension design step may occur at any desired point in the algorithm. The selection steps of the algorithm which are downstream of the additional extension design step may or may not include the additional residues in the analysis of the sequence.

Optionally the algorithm designs dsRNAs such that the optional additional extensions to the 19mer subsequences also have complementarity to the mRNA target region, however, this is not essential for the functionality of the dsRNA molecule.

In one embodiment, the algorithm generates one or more dsRNA molecules, which consist of the two strands stably base-paired together with a number of unpaired nucleotides at the 3′ end of each strand forming 3′ overhangs. The number of unpaired nucleotides forming the 3′ overhang of each strand is preferably in the range of 1 to 5 nucleotides, more preferably 1 to 3 nucleotides and most preferably 2 nucleotides. Typically, the algorithm generates dsRNA molecules containing about 19 to 30 base-pairs, preferably 19 to 23 base-pairs, most preferably 21 base-pairs.

Alternatively, the algorithm generates one or more “Dicer-substrate siRNAs” (D-siRNAs). The endonuclease Dicer is an important factor in miRNA biogenesis and siRNAs designed as Dicer substrates can have drastically increased potency compared to standard length siRNAs and shRNAs

D-siRNAs are asymmetric siRNA-duplexes in which the strands are between 22 and 30 nucleotides in length. Typically, one strand (the passenger strand) is 22 to 28 nucleotides long, preferably 25 nucleotides long, and the other strand (the guide strand) is 24 to 30 nucleotides long, preferably 27 nucleotides long, such that the duplex at the 3′ end of the passenger strand is blunt-ended and the duplex has an overhang on the 3′ end of the guide strand. The overhang is 1 to 3 nucleotides in length, preferably 2 nucleotides. The passenger strand may also contain a 5′ phosphate.

In the present invention the algorithm designs dsRNAs in which both strands function as guide strands and can be incorporated into RISC to function in RNAi, however, for the purposes of clarity, from hereon the shorter, typically 25-nucleotide strand of a D-siRNA will be referred to as the passenger strand or the 25mer strand and the longer, typically 27-nucleotide long strand will be referred to as the guide strand or the 27mer strand. The terms “25mer” and “27mer” are not intended to be limiting with regard to the length of the strands.

Typically in D-siRNAs, the two nucleotides at the 3′ end of the passenger strand are deoxyribonucleic acids (DNAs) rather than ribonucleic acids (RNAs). The DNAs and the blunt-ended duplex ensure that the enzyme Dicer processes the duplex into a 21 mer duplex consisting of the 21 nucleotides at the 5′ and 3′ ends of the original D-siRNA's passenger and guide strands respectively.

The (typically) 19mer bi-functional dsRNAs generated by the above discussed algorithm comprise two strands which are both guide strands since they both have complementarity to a target mRNA sequence and function in cleavage-mediate RNAi. The 19mer dsRNAs generated as a result of steps (i) to (iv) above can optionally be extended into D-siRNAs as follows:

(v) From the list of sorted candidate 19mer dsRNAs of step (iv), select the dsRNAs with the desired potential to cause translational suppression;

(vi) For each dsRNA selected in step (v), nominate one of the two 19mer strands to form a 27mer strand and add two nucleotides to the strand's 3′ end and six nucleotides to the strand's 5′ end, wherein the additional nucleotides are the reverse complements of the two and six nucleotides 5′ and 3′ respectively of the strand's 19mer target mRNA site;

(vii) Nominate the other strand of the dsRNA selected in step (v) to form a 25mer strand and to the 3′ end of this strand add the reverse complement of the 27mer strand's six 5′ nucleotides; and

(viii) Modify the two nucleotides at the 3′ end of the 25mer strand to DNAs instead of RNAs.

The D-siRNAs designed by the above algorithm will therefore consist of a 27mer strand and a 25mer strand. Preferably, all 27 nucleotides of the 27mer strand will have perfect complementarity to its cleavage mRNA target, whereas only the 19 nucleotides at the 5′ end of the 25mer long strand will have perfect complementarity to its cleavage target. However, the 25mer's reduced complementarity should not influence its potency since it still comprises the 19mer sequence with complementarity to the mRNA target sequence and so will still form the active functional siRNA (dsRNA) after Dicer processing.

When extended to 27mer/25mer D-siRNAs, many of the siRNA molecules have an end structure where the predicted number of unpaired bases at the 3′ end of the passenger strand is less than or equal to the predicted number of unpaired bases at the 5′ end of the guide strand. Based on the structure of known miRNAs and the binding requirements of the Dicer PAZ-domain, this structure is most likely suboptimal for Dicer processing and so, while useful as siRNA molecules, such duplexes are less useful when extended to Dicer-substrate siRNA molecules. Therefore, the algorithm optionally comprises an additional step in which D-siRNAs with such a predicted structure are identified and deselected.

The algorithm and methods of the present invention can optionally comprise one or more additional steps in which modifications to the dsRNA molecules are designed. For instance, the two strands of the dsRNA molecule may be linked by a linking component such as a chemical linking group or an oligonucleotide linker with the result that the resulting structure of the dsRNA is a hairpin structure. The linking component must not block or otherwise negatively affect the activity of the dsRNA, for instance by blocking loading of strands into the RISC complex or association with Dicer. Many suitable chemical linking groups are known in the art. If an oligonucleotide linker is used, it may be of any sequence or length provided that full functionality of the dsRNA is retained. Preferably, the linker sequence contains higher amounts of uridines and guanines than other nucleotide bases and has a preferred length of about 4 to 9, more preferably 8 or 9 residues.

Modifications can be included in the dsRNA, provided that the modification does not prevent the dsRNA composition from serving as a substrate for Dicer. One or more modifications can be made that enhance Dicer processing of the dsRNA, that result in more effective RNAi generation, that support a greater RNAi effect, that result in greater potency per each dsRNA molecule to be delivered to the cell and/or that are helpful in ensuring dsRNA stability in a therapeutic setting.

Modifications can be incorporated in the 3′-terminal region, the 5′-terminal region, in both the 3′-terminal and 5′-terminal region or in some instances in various positions within the sequence. With the restrictions noted above in mind any number and combination of modifications can be incorporated into the dsRNA. Where multiple modifications are present, they may be the same or different. Modifications to bases, sugar moieties, the phosphate backbone, and their combinations are contemplated. Either 5′-terminus can be phosphorylated.

The antisense strand can be modified for Dicer processing by suitable modifiers located at the 3′ end of the antisense strand, i.e., the dsRNA is designed to direct orientation of Dicer binding and processing. Suitable modifiers include nucleotides such as deoxyribonucleotides, dideoxyribonucleotides, acyclonucleotides and the like and sterically hindered molecules, such as fluorescent molecules and the like. Acyclonucleotides substitute a 2-hydroxyethoxymethyl group for the 2′-deoxyribofuranosyl sugar normally present in dNMPs. Other nucleotide modifiers could include 3′-deoxyadenosine (cordycepin), 3′-azido-3′-deoxythymidine (AZT), 2′,3′-dideoxyinosine (ddI), thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T). Deoxynucleotides can be used as the modifiers. When nucleotide modifiers are utilized, 1-3 nucleotide modifiers, or 2 nucleotide modifiers are substituted for the ribonucleotides on the 3′ end of the antisense strand. When sterically hindered molecules are utilized, they are attached to the ribonucleotide at the 3′ end of the antisense strand. Thus, the length of the strand does not change with the incorporation of the modifiers. The invention contemplates substituting two DNA bases in the dsRNA to direct the orientation of Dicer processing. In a further invention, two terminal DNA bases are located on the 3′ end of the antisense strand in place of two ribonucleotides forming a blunt end of the duplex on the 5′ end of the sense strand and the 3′ end of the antisense strand, and a two-nucleotide RNA overhang is located on the 3′-end of the sense strand. This is an asymmetric composition with DNA on the blunt end and RNA bases on the overhanging end.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1, each incorporated herein by reference. Other modifications are disclosed in Herdewijn (2000) Antisense Nucleic Acid Drug Dev 10: 297-310, Eckstein (2000) Antisense Nucleic Acid Drug Dev 10: 117-21, Rusckowski et al. (2000) Antisense Nucleic Acid Drug Dev 10: 333-345, Stein et al., (2001) Antisense Nucleic Acid Drug Dev 11: 317-325 and Vorobjev et al. (2001) Antisense Nucleic Acid Drug Dev 11: 77-85, each incorporated herein by reference.

The dsRNA molecules designed by the algorithm of the invention can be produced by any suitable method, for example synthetically or by expression in cells using standard molecular biology techniques which are well-known to the skilled artisan.

In a further aspect the present invention provides a method of designing a double-stranded RNA molecule which comprises performing an algorithm as defined above.

In a yet further aspect the present invention provides a method of producing a double stranded RNA molecule which comprises performing an algorithm as defined above and then synthesising one or more of the RNA molecules generated by said algorithm.

The dsRNAs of the invention can be obtained using a number of techniques known to those of skill in the art. For example, the dsRNAs can be chemically synthesized or recombinantly produced using methods known in the art, such as the Drosophila in vitro system described in U.S. published application 2002/0086356 of Tuschl et al., or the methods of synthesizing RNA molecules described in Verma and Eckstein (1998) Annu Rev Biochem 67: 99-134, the entire disclosures of which are herein incorporated by reference. The dsRNAs of the invention may be chemically synthesized using appropriately protected ribonucleoside phosphoramidites and a conventional DNA/RNA synthesizer. The dsRNAs can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

The dsRNAs can also be expressed from recombinant circular or linear DNA plasmids using any suitable promoter. Suitable promoters for expressing dsRNAs of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the dsRNA in a particular tissue or in a particular intracellular environment.

The dsRNAs expressed from recombinant plasmids can either be isolated from cultured cell expression systems by standard techniques, or can be expressed intracellularly at or near the area of disease in vivo. The dsRNAs of the invention can be expressed from a recombinant plasmid either as two separate, complementary RNA molecules, or as a single RNA molecule with two complementary regions.

Selection of plasmids suitable for expressing dsRNAs of the invention, methods for inserting nucleic acid sequences for expressing the dsRNAs into the plasmid, and methods of delivering the recombinant plasmid to the cells of interest are within the skill in the art. See, for example Tuschl, T. (2002), Nat. Biotechnol. 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference.

The dsRNAs of the invention can also be expressed from, recombinant viral vectors intracellularly in vivo. The recombinant viral vectors of the invention comprise sequences encoding the dsRNAs of the invention and any suitable promoter for expressing the dsRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the dsRNAs in a particular tissue or in a particular intracellular environment. dsRNAs of the invention can be expressed from a recombinant viral vector either as two separate, complementary RNA molecules, or as a single RNA molecule with two complementary regions. Any viral vector capable of accepting the coding sequences for the dsRNAs molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; and Anderson W F (1998), Nature 392: 25-30, the entire disclosures of which are herein incorporated by reference.

The ability of a dsRNA containing a given target sequence to cause RNAi-mediated degradation of the target mRNA can be evaluated using standard techniques for measuring the levels of RNA or protein in cells. For example, dsRNA of the invention can be delivered to cultured cells, and the levels of target mRNA can be measured by Northern blot or dot blotting techniques, or by quantitative RT-PCR. Alternatively, the levels of VEGF, Flt-1 or Flk-1/KDR receptor protein in the cultured cells can be measured by ELISA or Western blot.

Using the above algorithm, the present inventors have designed specific dsRNA molecules which effectively modulate the activity of numerous target genes associated with prostate cancer.

In a preferred embodiment, the genes targeted for down-regulation, i.e. the genes of interest from which the population of mRNA sequences in step (i) of the above algorithm are transcribed, are selected from the genes shown in Table 1, which are implicated in the initiation or progression of prostate cancer.

TABLE 1
Details of seven prostate cancer-related oncogenes

| Target Gene ID | RefSeq ID | mRNA length | 3′ UTR length |
|---|---|---|---|
| ERG1 | NM_182918 | 3057 | 1524 |
| cMyc | NM_002467 | 2356 | 467 |
| ERG2 | NM_004449 | 3099 | 1524 |
| BCL2 | NM_000633 | 6492 | 5279 |
| hTERT | NM_198253 | 4013 | 556 |
| ETV1 | NM_004956 | 6154 | 4633 |
| EGFRa | NM_005228 | 5599 | 1720 |

As discussed in Examples 1 and 5, using the mRNA sequences transcribed from the genes of interest in the table above, the inventors used the algorithm discussed above to identify stable 19mer multi-targeting dsRNA molecules in which both strands had complementarity to the coding sequence(s) of at least one of the target mRNA sequences and in which at least one strand, preferably both strands had one or more seed region(s) complementary to one or more seed sites in at least one of the target mRNA sequences. Both strands of the dsRNAs identified function in siRNA-like downregulation of the target genes whose mRNA transcripts they have complementarity to and at least one strand, preferably both strands also function in miRNA-like downregulation of the target genes whose mRNA transcripts they have complementarity to.

In a preferred embodiment, the double-stranded RNA molecules of the invention can target mRNA corresponding to one or more prostate cancer-related oncogenes, preferably one or more of the oncogenes in Table 1, preferably 2 or more of these oncogenes.

Thus, in a further aspect the present invention provides dsRNA molecules with the specific sequences shown in Table 2.

TABLE 2

Top-scoring multifunctional siRNAs against all combinations of
ERG1, MYC, ERG2, BCL2, hTERT, ETV1, and EGFRa. The table
lists the dsRNA ID, the target gene IDs and the 5′ to 3′
sequences of both strands of the dsRNA

| | |
|---|---|
| Strand 1 | Strand 2 |

| dsRNA | Target | Strand Sequence | Target | Strand Sequence |
|---|---|---|---|---|
| Duplex ID | Gene ID | 5′ -> 3′ | Gene ID | 5′ -> 3′ |
| PCS1 | EGFRa | caaacaagucagucuaacg | BCL2 | ccuuagaauggcuuguauu |
| PCS2 | ETV1 | auuucucccacgcugacua | BCL2 | uaagugagcuguggagaga |
| PCS3 | ETV1 | uuuaguggaauaguugaaa | BCL2 | auuucaacugacucccuaa |
| PCS4 | ETV1 | caauucucuguaucuugca | cMYC | uggcaauauuuacagagaa |
| PCS5 | ETV1 | guuucaugacuuaguguuu | ERG1 | auaacacugacugcaugaa |
| PCS6 | ETV1 | guuucaugacuuaguguuu | ERG2 | auaacacugacugcaugaa |
| PCS7 | ETV1 | ucaaacagacaugauauag | ERG1 | cauaucaacgucuguugau |
| PCS8 | ETV1 | ucaaacagacaugauauag | ERG2 | cauaucaacgucuguugau |
| PCS9 | EGFRa | aaauucacugcuuuguggc | ETV1 | gacagcaaacagcagugaa |
| PCS10 | BCL2 | guuucuuacucagacagag | ERG1 | cucauuucuguaguaagac |
| PCS11 | BCL2 | guuucuuacucagacagag | ERG2 | cucauuucuguaguaagac |
| PCS12 | BCL2 | cuuuagaugauaagcauuu | cMYC | aaaucuuucagucucaaga |
| PCS13 | ETV1 | ccuguuacacagaauaaau | cMYC | auugaaauucuguguaacu |
| PCS14 | ETV1 | augaaaucaaacagacaug | hTERT | caagugcugucugauucca |
| PCS15 | hTERT | aggccucggccaaacacuc | BCL2 | gaguauuugugcagcgagg |
| PCS16 | ETV1 | aagccauguuuccuguaau | cMYC | auuuacagagaaacauugu |
| PCS17 | EGFRa | cugauuucaugacagcaag | ETV1 | cuuauguuguuaugaaauc |
| PCS18 | EGFRa | gccgaagggcaugagcugc | hTERT | gacacucagcccuuggcug |
| PCS19 | EGFRa | gguggcugccuuucugggc | BCL2 | ggcccagauaggcacccag |
| PCS20 | BCL2 | caaacggagcugcacuuug | ERG1 | caaaugcagacuccuguau |
| PCS21 | EGFRa | cuguuaaccagucagguua | ERG1 | uaacacugacugcaugaac |
| PCS22 | EGFRa | cuguuaaccagucagguua | ERG2 | uaacacugacugcaugaac |
| PCS23 | hTERT | ucaggccucagacucccag | cMYC | cucgguguccgaggaccug |
| PCS24 | cMYC | auaggugauugcucaggac | ERG1 | guuccuugagccauucacc |
| PCS25 | hTERT | cagggcacgcacaccaggc | ERG1 | gccagguguggcguuccgu |
| PCS26 | ERG2 | acaggagcuccaggaggaa | cMYC | ucuccucauggagcaccag |
| PCS27 | EGFRa | uguugugaaagcauuauca | cMYC | ugaucaugcauuugaaaca |
| PCS28 | hTERT | ccaaagagguggcuucuuc | ERG2 | gccaagaaggccaucucuu |
| PCS29 | ETV1 | aucuugagauggagagauu | cMyc | aaucuuucagucucaagac |
| PCS30 | EGFRa | gaauagaugacucaaggca | ETV1 | ugguuugagucaucuauuu |
| PCS31 | cMyc | caaguccucuucagaaaug | ERG1 | cauuucuuaagaccacuuu |

The invention also provides single-stranded (ss)RNA molecules comprising or consisting of the above individual strand sequences.

The invention also provides DNA molecules equivalent to the above mentioned RNA molecules.

As discussed in Examples 2, 4 and 6, the above siRNA molecules were extended to D-siRNA molecules in accordance with the algorithm discussed above. Accordingly in a further aspect of the invention, dsRNA molecules with the following specific sequences set out in Table 3 are provided.

TABLE 3
Dicer substrate siRNAs based on the 19mer duplexes in Table 2. The table
shows the dicer substrate strands and the two strands' duplex as
predicted by RNAhybrid. DNAs are in uppercase whereas RNAs
are in lower case.

| dsRNA | Target | | |
|---|---|---|---|
| Duplex | Gene | Strand Sequences | |
| ID | IDs | 5′ → 3′ | Duplex Structure |
| PCS1 | EGFRa BCL2 | caaacaagucagucuaacgaaauTA uaauuuccuuagaauggcuuguauuuc | 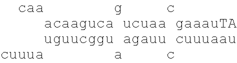 |
| PCS2 | ETV1 BCL2 | auuucucccacgcugacuaccucTC gagagguaagugagcuguggagagaau | 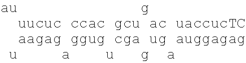 |
| PCS3 | ETV1 BCL2 | uuuaguggaauaguugaaaucuaTT aauagaauuucaacugacucccuaauu | 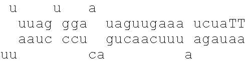 |
| PCS4 | ETV1 cMYC | caauucucuguaucuugcauuaaAT auuuaauggcaauauuuacagagaaac | 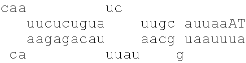 |
| PCS5 | ETV1 ERG1 | guuucaugacuuaguguuuaccaAA uuugguauaacacugacugcaugaacc | 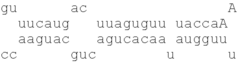 |
| PCS6 | ETV1 ERG2 | guuucaugacuuaguguuuaccaAA uuugguauaacacugacugcaugaacc | 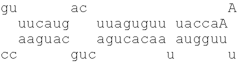 |
| PCS7 | ETV1 ERG1 | ucaaacagacaugauauagcaacTG caguugcauaucaacgucuguugaugg | 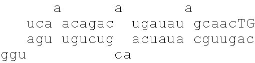 |
| PCS8 | ETV1 ERG2 | ucaaacagacaugauauagcaacTG Caguugcauaucaacgucuguugaugg | 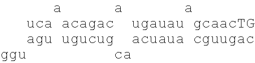 |
| PCS9 | EGFRa ETV1 | aaauucacugcuuuguggcucuuGA ucaagagacagcaaacagcagugaaga | 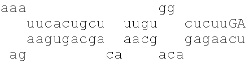 |
| PCS10 | BCL2 ERG1 | guuucuuacucagacagaggaggAT auccuccucauuucuguaguaagacuu | 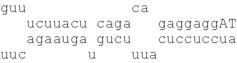 |
| PCS11 | BCL2 ERG2 | guuucuuacucagacagaggaggAT auccuccucauuucuguaguaagacuu | 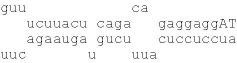 |
| PCS12 | BCL2 cMYC | cuuuagaugauaagcauuuagccAT auggcuaaaucuuucagucucaagacu | 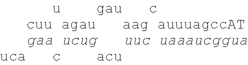 |
| PCS13 | ETV1 cMYC | ccuguuacacagaauaaauccuaGT acuaggauugaaauucuguguaacugc | 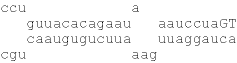 |
| PCS14 | ETV1 hTERT | augaaaucaaacagacaugaagaGG ccucuucaagugcugucugauuccaau | 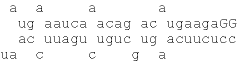 |
| PCS15 | hTERT BCL2 | aggccucggccaaacacuccgcaAG cuugcggaguauuugugcagcgaggga | 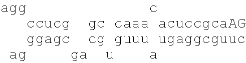 |
| PCS16 | ETV1 cMYC | aagccauguuuccuguaauauugCC ggcaauauuuacagagaaacauugugu | 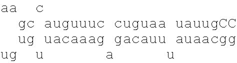 |
| PCS17 | EGFRa ETV1 | cugauuucaugacagcaagauaaTG cauuaucuuauguuguuaugaaaucaa | 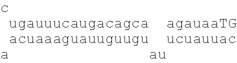 |
| PCS18 | EGFRa hTERT | gccgaagggcaugagcugccagcAC gugcuggacacucagcccuuggcugga | 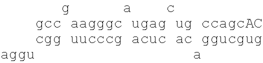 |
| PCS19 | EGFRa BCL2 | gguggcugccuuucugggcacaaGT Acuuguggcccagauaggcacccaggg | 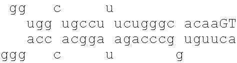 |
| PCS20 | BCL2 ERG1 | caaacggagcugcacuuugcacuTT aaagugcaaaugcagacuccuguauaa | 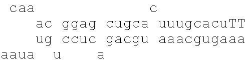 |
| PCS21 | EGFRa ERG1 | cuguuaaccagucagguuauaccAA uugguauaacacugacugcaugaaccc | 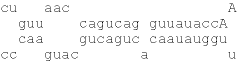 |
| PCS22 | EGFRa ERG2 | cuguuaaccagucagguuauaccAA uugguauaacacugacugcaugaaccc | 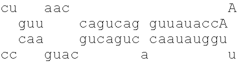 |
| PCS23 | hTERT cMYC | ucaggccucagacucccaggagaAT auucuccucgguguccgaggaccuggg | 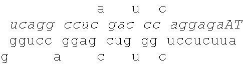 |
| PCS24 | cMYC ERG1 | auaggugauugcucaggacucucCT aggagaguuccuugagccauucaccug | 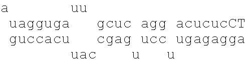 |
| PCS25 | hTERT ERG1 | cagggcacgcacaccaggcuaagAC gucuuagccagguguggcguuccguag | 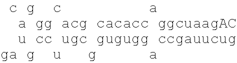 |
| PCS26 | ERG2 cMYC | acaggagcuccaggaggaacaccGC gcggugucuccucauggagcaccaggg | 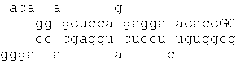 |
| PCS27 | EGFRa cMYC | uguugugaaagcauuaucaaaugCA ugcauuugaucaugcauuugaaacaag | 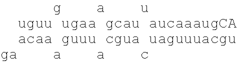 |
| PCS28 | hTERT ERG2 | ccaaagagguggcuucuucugccAC guggcagccaagaaggccaucucuuac | 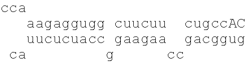 |
| PCS29 | ETV1 cMYC | aucuugagauggagagauuuagcCA uggcuaaaucuuucagucucaagacuc | 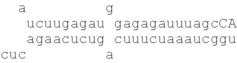 |
| PCS30 | EGFRa ETV1 | gaauagaugacucaaggcauuuaTT aauaaaugguuugagucaucuauuuug | 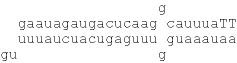 |
| PCS31 | cMyc ERG1 | caaguccucuucagaaauguauaAA uuuauacauuucuuaagaccacuuucu | 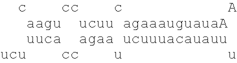 |

Preferably, the dsRNA molecules of the present invention are those which, when extended to 27mer Dicer substrate siRNAs do not have an end structure where the predicted number of unpaired bases at the 3′ end of the bottom strand as shown in the duplex structure in Table 3 above is less than or equal to the predicted number of unpaired bases at the 5′ end of the top strand. Such dsRNA molecules which do not have this structure are less likely to be suboptimal for Dicer processing. Thus, in a preferred embodiment, the present invention provides dsRNA molecules with the specific sequences shown in Table 4:

TABLE 4

Top-scoring multifunctional siRNAs against all combinations
of ERG1, MYC, ERG2, BCL2, hTERT, ETV1, and EGFRa.
The table lists the dsRNA ID, the target gene IDs and the
5′ to 3′ sequences of both strands of the dsRNA. These
dsRNAs were selected from the top-scoring multi-targeting
siRNAs (Table 2) based on their duplex end structure,
such that the predicted number of unpaired bases at the
3′ end of the bottom strand is greater than the predicted
number of unpaired bases at the 5′ end of the top strand

| | |
|---|---|
| Strand 1 | Strand 2 |

| dsRNA | Target | | Target | |
|---|---|---|---|---|
| Duplex | Gene | Strand Sequence | Gene | Strand Sequence |
| ID | ID | 5′ -> 3′ | ID | 5′ -> 3′ |
| PCS1 | EGFRa | caaacaagucagucuaacg | BCL2 | ccuuagaauggcuuguauu |
| PCS3 | ETV1 | uuuaguggaauaguugaaa | BCL2 | auuucaacugacucccuaa |
| PCS7 | ETV1 | ucaaacagacaugauauag | ERG1 | cauaucaacgucuguugau |
| PCS12 | BCL2 | cuuuagaugauaagcauuu | cMYC | aaaucuuucagucucaaga |
| PCS14 | ETV1 | augaaaucaaacagacaug | hTERT | caagugcugucugauucca |
| PCS18 | EGFRa | gccgaagggcaugagcugc | hTERT | gacacucagcccuuggcug |
| PCS19 | EGFRa | gguggcugccuuucugggc | BCL2 | ggcccagauaggcacccag |
| PCS20 | BCL2 | caaacggagcugcacuuug | ERG1 | caaaugcagacuccuguau |
| PCS29 | ETV1 | aucuugagauggagagauu | cMyc | aaucuuucagucucaagac |
| PCS30 | EGFRa | gaauagaugacucaaggca | ETV1 | ugguuugagucaucuauuu |
| PCS31 | cMyc | caaguccucuucagaaaug | ERG1 | cauuucuuaagaccacuuu |

In a further preferred embodiment, the multi-targeting Dicer-substrate siRNA molecules with the following specific sequences shown in Table 5 are provided:

TABLE 5
Dicer substrate siRNAs based on the 19mer duplexes in Table 4. The table
shows the dicer substrate strands and the two strands' duplex as
predicted by RNAhydrid. DNSs are in uppercase whereas
RNAs are in lower case.

| dsRNA | Target | | |
|---|---|---|---|
| Duplex | Gene | Strand Sequences | |
| ID | IDs | 5′ → 3′ | Duplex Structure |
| PCS1 | EGFRa BCL2 | caaacaagucagucuaacgaaauTA uaauuuccuuagaauggcuuguauuuc | 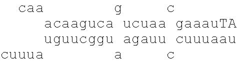 |
| PCS3 | ETV1 BCL2 | uuuaguggaauaguugaaaucuaTT aauagaauuucaacugacucccuaauu | 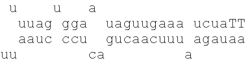 |
| PCS7 | ETV1 ERG1 | ucaaacagacaugauauagcaacTG caguugcauaucaacgucuguugaugg | 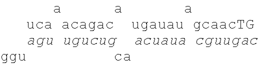 |
| PCS12 | BCL2 cMYC | cuuuagaugauaagcauuuagccAT auggcuaaaucuuucagucucaagacu | 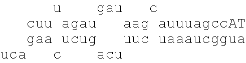 |
| PCS14 | ETV1 hTERT | augaaaucaaacagacaugaagaGG ccucuucaagugcugucugauuccaau | 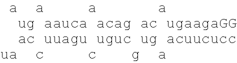 |
| PCS18 | EGFRa hTERT | gccgaagggcaugagcugccagcAC gugcuggacacucagcccuuggcugga | 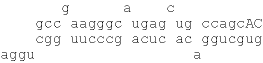 |
| PCS19 | EGFRa BCL2 | gguggcugccuuucugggcacaaGT acuuguggcccagauaggcacccaggg | 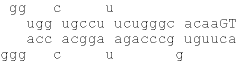 |
| PCS20 | BCL2 ERG1 | caaacggagcugcacuuugcacuTT aaagugcaaaugcagacuccuguauaa | 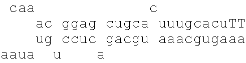 |
| PCS29 | ETV1 cMyc | aucuugagauggagagauuuagcCA uggcuaaaucuuucagucucaagacuc | 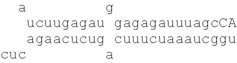 |
| PCS30 | EGFRa ETV1 | gaauagaugacucaaggcauuuaTT aauaaaugguuugagucaucuauuuug | 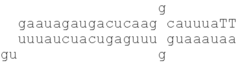 |
| PCS31 | cMyc ERG1 | caaguccucuucagaaauguauaAA uuuauacauuucuuaagaccacuuucu | 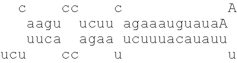 |

These specific multi-targeting dsRNAs discussed above each contain two strands, which between them can cleave two of the seven genes ERG1, cMYC, ERG2, BCL2, hTERT, ETV1, and EGFRa and can cause translational repression of one or both of these target genes.

The dsRNAs of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the dsRNA; modifications that make the dsRNA resistant to nuclease digestion (e.g., the use of 2′-substituted ribonucleotides or modifications to the sugar-phosphate backbone); or the substitution of one or more nucleotides in the dsRNA with deoxyribonucleotides. Modifications can be made for instance so that one particular strand is preferentially loaded into RISC, with the proviso that both strands are capable of being loaded to some extent, i.e. with the proviso that no strand is exclusively loaded and no strand is never loaded. Such modifications are well-known to the skilled man.

The dsRNA molecules discussed above have utility as safe, effective, therapeutic and/or prophylactic agents, either alone or as adjuvants, to treat or prevent diseases, preferably cancer and cancer related diseases, most preferably prostate cancer and prostate cancer related diseases, in humans and animals by down-regulating the expression of one or more genes implicated in such diseases.

Thus, in a further aspect, the present invention provides the dsRNA molecules of the invention, and provided by the methods of the invention, for use in therapy.

In a further aspect, the present provides the specific dsRNA molecules of the invention, and provided by the methods of the invention, for use in the treatment of cancer and cancer related diseases.

Alternatively viewed, the invention provides the use of the dsRNA molecules of the invention, and provided by the methods of the invention, in the manufacture of a medicament for the treatment of cancer and cancer related diseases.

Alternatively viewed, the invention provides methods of treating cancer and cancer related diseased comprising the administration of an effective amount of the dsRNA molecules of the invention, and provided by the methods of the invention, to a subject.

Typically, the cancer is prostate cancer. The term “cancer related diseases” includes but is not limited to secondary diseases and conditions associated with the onset and progression of cancer as well as symptoms of cancer.

In preferred embodiments, the above therapeutic uses and methods utilise one or more of the specific RNA molecules recited herein, i.e. in Tables 2-11.

By “an effective amount” is meant an amount of a compound effective to ameliorate the symptoms of, or ameliorate, treat, prevent, delay the onset of or inhibit the progression of a disease. Ultimately, the attending physician or veterinarian will decide the appropriate amount and dosage regimen. The “effective amount” of the active ingredients that may be combined with the carrier materials to produce a single dosage will vary depending upon the subject treated and the particular mode of administration.

Typically the “subject” will be an animal, preferably a human in need of therapy, for instance due to being diagnosed with or being at risk of cancer, especially prostate cancer.

One skilled in the art can readily determine an effective amount of the dsRNAs of the invention to be administered to a given subject, by taking into account factors such as the size, weight, age, health and sex of the subject; the extent of the disease; the route of administration; and whether the administration is regional or systemic.

For treating prostate cancer, the dsRNAs of the invention can be administered to a subject in combination with an active pharmaceutical agent which is different from the present dsRNAs. Alternatively, the dsRNAs can be administered to a subject in combination with another therapeutic method designed to treat the prostate cancer, including but not limited to radiation therapy, chemotherapy, and surgery. For treating tumors, the dsRNAs of the invention are preferably administered to a subject in combination with radiation therapy, or in combination with chemotherapeutic agents such as cisplatin, carboplatin, cyclophosphamide, 5-fluorouracil, adriamycin, daunorubicin or tamoxifen.

In the present methods, the dsRNAs can be administered to the subject either as naked dsRNAs, in conjunction with a delivery reagent, or as a recombinant plasmid or viral vector which expresses the dsRNAs.

Suitable delivery reagents for administration in conjunction with the present dsRNAs include the Mirus Transit TKO lipophilic reagent; lipofectin; lipofectamine; cellfectin; or polycations (e.g., polylysine), or liposomes. A preferred delivery reagent is a liposome. A variety of methods are known for preparing liposomes, for example as described in Szoka et al. (1980), Ann. Rev. Biophys. Bioeng, 9: 467; and U.S. Pat. Nos. 4,235,871 and 5,019,369, the entire disclosures of which are herein incorporated by reference.

Preferably, the liposomes encapsulating the present dsRNAs comprises a ligand molecule that can target the liposome to a particular cell or tissue at or near the site of the disease. Ligands which bind to receptors prevalent in prostate cancer are preferred.

Particularly preferably, the liposomes encapsulating the present dsRNAs are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand.

Recombinant plasmids which express the dsRNAs can also be administered directly or in conjunction with a suitable delivery reagent, including the Mirus Transit LT1 lipophilic reagent; lipofectin; lipofectamine; cellfectin; polycations (e.g., polylysine) or liposomes. Recombinant viral vectors which express the dsRNA and methods for delivering such vectors to an area of disease in a patient are within the skill in the art.

The dsRNAs of the invention can be administered to the subject by any means suitable for delivering the dsRNAs to the cells of the tissue at or near the area of disease. For example, the dsRNAs can be administered by gene gun, electroporation, or by other suitable parenteral or enteral administration routes. Suitable administration routes include oral, rectal, or intranasal deliver as well as intravascular administration (e.g. intravenous bolus injection, intravenous infusion, intra-arterial bolus injection, intra-arterial infusion and catheter instillation into the disease site); peri- and intra-tissue administration (e.g., peri-tumoral and intra-tumoral injection); subcutaneous injection or deposition including subcutaneous infusion (such as by osmotic pumps); direct application to the area at or near the site of disease, for example by a catheter or other placement device (e.g., a suppository or an implant comprising a porous, non-porous, or gelatinous material); and inhalation. It is preferred that injections or infusions of the dsRNA are given at or near the site of disease.

The dsRNAs of the invention can be administered in a single dose or in multiple doses. Where the administration is by infusion, the infusion can be a single sustained dose or can be delivered by multiple infusions.

One skilled in the art can readily determine an appropriate dosage regimen for administering the dsRNAs of the invention to a given subject. For example, the dsRNAs can be administered to the subject once, such as by a single injection or deposition at or near the disease site. Alternatively, the dsRNA can be administered to a subject once or twice daily to a subject for a period of time determinable by one skilled in the art. Where a dosage regimen comprises multiple administrations, it is understood that the effective amount of dsRNA administered to the subject can comprise the total amount of dsRNA administered over the entire dosage regimen.

The dsRNAs of the invention are preferably formulated as pharmaceutical compositions prior to administering to a subject.

Thus, in a further aspect, the present invention also provides a pharmaceutical composition comprising a dsRNA as defined above and a physiologically acceptable carrier, diluent or excipient.

Techniques for formulating pharmaceutical compositions are well-known in the art. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise one or more dsRNAs of the invention (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional non-toxic solid carriers can be used; for example, pharmaceutical grades of mannitol, lactose, starch, magnesium stearate, sodium saccharin, talcum, cellulose, glucose, sucrose, magnesium carbonate, and the like.

Nano-particles are also potential delivery vehicles. Sayin et al., (2008) International Journal of Pharmaceutics Vol. 363, Issues 1-2, pp 139-148, the contents of which are incorporated here by reference discloses such nano-particles which can be used in this aspect of the invention.

This invention also provides a kit or an administration device comprising a dsRNA as described herein and information material which describes administering the dsRNA to a human or other animal. The kit or administration device may have a compartment containing the dsRNA. As used herein, the “Information material” includes, but is not limited to, a publication, a recording, a diagram, or any other medium of expression which can be used to communicate the usefulness of the composition of the invention for its designated use.

The compositions described herein may comprise, consist essentially of, or consist of any of the elements as described herein.

Various documents including, for example, publications and patents, are recited throughout this disclosure. All such documents are, in relevant part, hereby incorporated by reference. The citation of any given document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

The following examples are intended to be illustrative of the present invention and to teach one of ordinary skill in the art to make and use the invention. These examples are not intended to limit the invention in any way. The invention will now be further described in the following Examples and the figures in which:

FIG. 1 is a schematic drawing of the structure of the dsRNAs. (A) Schematic diagram of the basic multi-targeting siRNA; red illustrates the dsRNA seed region and green illustrates the rest of the 19mer siRNA guide strand. (B) Multi-targeting siRNA designed to target one transcript that has one cleavage site somewhere within the mRNA and several seed sites within the 3′ UTR. (C) Schematic of guide-only multi-targeting (bi-functional) dsRNAs of the present invention; red and yellow illustrate the seed regions of both strands and green and blue illustrates the rest of the two 19mer guide strands. (D) Schematic diagram of the dsRNAs of the present invention. In this scheme, the two strands of the bi-functional siRNA have cleavage sites in two different mRNAs and several seed sites within the 3′ UTRs of the two mRNAs.

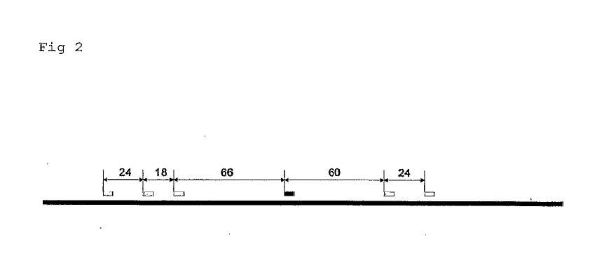

FIG. 2 is a schematic drawing of a mRNA 3′ UTR with six seed sites that group into three seed site modules (red, blue, and pink).

### EXAMPLES

### Example 1

A literature survey identified several genes, including ERG [40], ETV1 [41], hTERT [42, 43], MYC [44, 45], BCL2 [46], and EGFR [47], that are thought to contribute to the initiation or progression of prostate cancer which are shown in Table 1, see page repeated below:

TABLE 1

| Target Gene ID | RefSeq ID | mRNA length | 3′ UTR length |
|---|---|---|---|
| ERG1 | NM_182918 | 3057 | 1524 |
| cMyc | NM_002467 | 2356 | 467 |
| ERG2 | NM_004449 | 3099 | 1524 |
| BCL2 | NM_000633 | 6492 | 5279 |
| hTERT | NM_198253 | 4013 | 556 |
| ETV1 | NM_004956 | 6154 | 4633 |
| EGFRa | NM_005228 | 5599 | 1720 |

Using the algorithm of the present invention, several multi-targeting dsRNAs were designed where the two strands can cleave two of the seven genes ERG1, cMYC, ERG2, BCL2, hTERT, ETV1, and EGFRa and can cause translational repression of one or both of the target genes. These dsRNA molecules are shown in Table 6 below. The Table is sorted based on how likely the dsRNAs can cause translational suppression and show the one or two highest-scoring dsRNAs against each possible combination of the target genes.

In this design, a custom computer program was used that implements a version of the algorithm of the present invention. The program was written in the Python programming language (http://www.python.org/) and used RefSeq sequence IDs as input. The program starts by automatically downloading sequence annotations and sequence data from the UCSC Genome Browser Database (Karoichik et al., (2008) Nucleic Acids Research Vol. 36, Database Issue D773-339) and then automatically proceeds through the steps of the design algorithm. The different steps of the design algorithm were implemented as follows:

(i) Sequence input: The program uses a custom module that downloads the genomic locations of a given RefSeq sequence ID from the UCSC Genome Browser Database and then retrieves the gene's mRNA sequence by (a) mapping the genomic locations of its exons to a local copy of the hg18 version of the human genome and (b) joining the corresponding exon DNA sequences into a single sequence and translating this sequence into RNA.

(ii) Complementarity screen: The program uses a custom module that computes the edit distance between all pairs of 19mer subsequences within the input sequences. For this particular example, the maximum edit distance allowed was five (insertions, deletions, or substitutions).

(iii) Candidate list: The program uses a combination of custom modules and external programs to perform the four steps involved in generating the candidate list. More specifically, the program:

(a) uses a custom module to identify and remove each duplex that contains one or more of the motifs aaaa, cccc, gggg, and uuuu in either strand;

(b) uses a custom module to identify and remove each duplex that contains less than 25% or greater than 75% GCs in either strand;

(c) uses the external program described in (Snøve, O., Jr., et al. (2004) Biochem Biophys Res Commun 325: 769-773) to identify a dsRNA's potential off-target transcripts and then uses a custom module to remove each duplex where either strand had less than three mismatches to off-target transcripts; and

(d) uses the external program RNAfold to predict the difference in thermodynamic end stability by considering the 5 closing nucleotides at the ends of a 19mer duplex and removes each duplex where the absolute value of the difference is greater or equal to 3′ kcal/mol.

(iv) Sorted list: The program uses a custom module to search the two 3′ UTRs of each siRNA candidate's two target sequences and the uses a custom module that implements the previously described ranking algorithm to sort the siRNA candidates. In this particular example, the parameter used in the ranking algorithm were
Maximum distance between seeds in a seed site module: 35;

g(d₁)={0 if 13; 1 if dᵢ≧17; and (dⱼ−13)/4 otherwise}; and

h(dᵢ₋₁)={0.25 if dᵢ₋₁≧70; (70−dᵢ₋₁)/35 otherwise}.

TABLE 6

Top-scoring multifunctional siRNAs against all combinations of ERG1, MYC,
ERG2, BCL2, hTERT, ETV1, and EGFRa. The table lists dsRNA ID, the target
genes, the corresponding strand which targets that gene for siRNA-like
downregulation, the cleavage location in the target sequence, the predicted
thermodynamic end stability, the seed site locations in the target sequence's
3′ UTR and cleavage efficacy. The 3′UTR seed sites listed in the same
row as a target gene are those seed sites found in that target gene's 3′UTR
sequence. A given seed site may be complementary to the seed region of either
strand of the dsRNA. Efficacy is the predicted cleavage efficacy of a
“standard” dsRNA designed against the corresponding target sites;
values >0 indicate effective dsRNAs with larger values representing more
confident predictions. Horizontal lines separate the different siRNA duplexes.

| | | | | | | |
|---|---|---|---|---|---|---|
| dsRNA | | | | | | |
| Duplex | Target | Strand Sequence | Cleavage | End | | |
| ID | Gene ID | (5′--> 3′) | Location | Stability | 3′ UTR Seed Sites | Efficacy |

| | | | | | | |
|---|---|---|---|---|---|---|
| PCS1 | EGFRa | caaacaagucagucuaacg | 4600 | −3.7 | [cuuguuu:7mer@732, | 0.05 |
| | | | | | uuguuu:6mer@752] | |
| | BCL2 | ccuuagaauggcuuguauu | 4999 | −2.2 | [uuguuu:6mer@1833, | 0.0 |
| | | | | | uuguuu:6mer@2006, | |
| | | | | | uuguuu:6mer@2046, | |
| | | | | | cuuguuu:7mer@2084, | |
| | | | | | ucuaag:6mer@2840, | |
| | | | | | uuguuu:6mer@3433, | |
| | | | | | uucuaag:7mer@3797, | |
| | | | | | uuguuu:6mer@4892, | |
| | | | | | uuguuu:6mer@4934, | |
| | | | | | uuguuu:6mer@4959, | |
| | | | | | uuguuu:6mer@5182] | |
| PCS2 | ETV1 | auuucucccacgcugacua | 131 | −3.7 | [ucacuu:6mer@878, | 0.32 |
| | | | | | ggagaaa:7mer@1517, | |
| | | | | | ggagaaa:7mer@2156, | |
| | | | | | ucacuu:6mer@3046, | |
| | | | | | ucacuu:6mer@3088, | |
| | | | | | gagaaa:6mer@3921, | |
| | | | | | cucacuu:7mer@4265, | |
| | | | | | ucacuu:6mer@4517, | |
| | | | | | gagaaa:6mer@4551] | |
| | BCL2 | uaagugagcuguggagaga | 4585 | −5.1 | [ucacuu:6mer@483, | 0.04 |
| | | | | | gagaaa:6mer@737, | |
| | | | | | cucacuu:7mer@3383, | |
| | | | | | ucacuu:6mer@3729, | |
| | | | | | gagaaa:6mer@3764, | |
| | | | | | gagaaa:6mer@4164] | |
| PCS3 | ETV1 | uuuaguggaauaguugaaa | 5024 | −2.1 | [cacuaa:6mer@991, | 0.32 |
| | | | | | uugaaa:6mer@1463, | |
| | | | | | uugaaa:6mer@1527, | |
| | | | | | uugaaa:6mer@1557, | |
| | | | | | guugaaa:7mer@2457, | |
| | | | | | uugaaa:6mer@2484, | |
| | | | | | uugaaa:6mer@3415, | |
| | | | | | uugaaa:6mer@3482, | |
| | | | | | ccacuaa:7mer@3514, | |
| | | | | | cacuaa:6mer@3583] | |
| | BCL2 | auuucaacugacucccuaa | 5021 | −1.2 | [cacuaa:6mer@38, | 0.44 |
| | | | | | ccacuaa:7mer@302, | |
| | | | | | uugaaa:6mer@1512, | |
| | | | | | uugaaa:6mer@1684, | |
| | | | | | uugaaa:6mer@2692, | |
| | | | | | guugaaa:7mer@3819, | |
| | | | | | uugaaa:6mer@5000] | |
| PCS4 | ETV1 | caauucucuguaucuugca | 1567 | −1.5 | [gagaauu:7mer@57, | 0.14 |
| | | | | | agaauu:6mer@917, | |
| | | | | | agaauu:6mer@2295, | |
| | | | | | uauugcc:7mer@3830, | |
| | | | | | agaauu:6mer@3846, | |
| | | | | | agaauu:6mer@4056, | |
| | | | | | agaauu:6mer@4143, | |
| | | | | | gagaauu:7mer@4319, | |
| | | | | | agaauu:6mer@4370] | |
| | cMYC | uggcaauauuuacagagaa | 2146 | −3.3 | [agaauu:6mer@219, | 0.04 |
| | | | | | uauugcc:7mer@268, | |
| | | | | | agaauu:6mer@319] | |
| PCS5 | ETV1 | guuucaugacuuaguguuu | 5101 | −1.5 | [augaaa:6mer@307, | 0.15 |
| | | | | | guguua:6mer@1060, | |
| | | | | | caugaaa:7mer@1762, | |
| | | | | | augaaa:6mer@2193, | |
| | | | | | caugaaa:7mer@2797, | |
| | | | | | caugaaa:7mer@3591, | |
| | | | | | guguua:6mer@3685, | |
| | | | | | augaaa:6mer@4564] | |
| | ERG1 | auaacacugacugcaugaa | 2359 | −1.5 | [augaaa:6mer@94, | 0.21 |
| | | | | | augaaa:6mer@358, | |
| | | | | | aguguua:7mer@837, | |
| | | | | | aguguua:7mer@854, | |
| | | | | | augaaa:6mer@944, | |
| | | | | | augaaa:6mer@1425, | |
| | | | | | guguua:6mer@1456] | |
| PCS6 | ETV1 | guuucaugacuuaguguuu | 5101 | −1.5 | [augaaa:6mer@307, | 0.15 |
| | | | | | guguua:6mer@1060, | |
| | | | | | caugaaa:7mer@1762, | |
| | | | | | augaaa:6mer@2193, | |
| | | | | | caugaaa:7mer@2797, | |
| | | | | | caugaaa:7mer@3591, | |
| | | | | | guguua:6mer@3685, | |
| | | | | | augaaa:6mer@4564] | |
| | ERG2 | auaacacugacugcaugaa | 2401 | −1.5 | [augaaa:6mer@94, | 0.21 |
| | | | | | augaaa:6mer@358, | |
| | | | | | aguguua:7mer@837, | |
| | | | | | aguguua:7mer@854, | |
| | | | | | augaaa:6mer@944, | |
| | | | | | augaaa:6mer@1425, | |
| | | | | | guguua:6mer@1456] | |
| PCS7 | ETV1 | ucaaacagacaugauauag | 1834 | −1.6 | [uguuug:6mer@88, | 0.3 |
| | | | | | cuguuug:7mer@324, | |
| | | | | | cuguuug:7mer@563, | |
| | | | | | uugauau:7mer@1032, | |
| | | | | | ugauau:6mer@1341, | |
| | | | | | ugauau:6mer@1410, | |
| | | | | | uguuug:6mer@1460, | |
| | | | | | cuguuug:7mer@2693, | |
| | | | | | uguuug:6mer@3954] | |
| | ERG1 | cauaucaacgucuguugau | 2028 | −1.3 | [uugauau:7mer@506, | 0.09 |
| | | | | | uguuug:6mer@934, | |
| | | | | | ugauau:6mer@968, | |
| | | | | | uguuug:6mer@1512] | |
| PCS8 | ETV1 | ucaaacagacaugauauag | 1834 | −1.6 | [uguuug:6mer@88, | 0.3 |
| | | | | | cuguuug:7mer@324, | |
| | | | | | cuguuug:7mer@563, | |
| | | | | | uugauau:7mer@1032, | |
| | | | | | ugauau:6mer@1341, | |
| | | | | | ugauau:6mer@1410, | |
| | | | | | uguuug:6mer@1460, | |
| | | | | | cuguuug:7mer@2693, | |
| | | | | | uguuug:6mer@3954] | |
| | ERG2 | cauaucaacgucuguugau | 2070 | −1.3 | [uugauau:7mer@506, | 0.09 |
| | | | | | uguuug:6mer@934, | |
| | | | | | ugauau:6mer@968, | |
| | | | | | uguuug:6mer@1512] | |
| PCS9 | EGFRa | aaauucacugcuuuguggc | 3848 | 0.0 | [uugcugu:7mer@783] | 0.35 |
| | ETV1 | gacagcaaacagcagugaa | 4207 | −2.6 | [uugcugu:7mer@560, | −0.2 |
| | | | | | uugcugu:7mer@734, | |
| | | | | | ugcugu:6mer@945, | |
| | | | | | ugcugu:6mer@1056, | |
| | | | | | ugcugu:6mer@1110, | |
| | | | | | ugaauu:6mer@1142, | |
| | | | | | uugcugu:7mer@1385, | |
| | | | | | ugaauu:6mer@1628, | |
| | | | | | uugcugu:7mer@2381, | |
| | | | | | ugaauu:6mer@2609, | |
| | | | | | ugaauu:6mer@2621, | |
| | | | | | ugcugu:6mer@2691, | |
| | | | | | uugcugu:7mer@2697, | |
| | | | | | ugcugu:6mer@2894] | |
| PCS10 | BCL2 | guuucuuacucagacagag | 2054 | −3.2 | [uaagaaa:7mer@103, | 0.25 |
| | | | | | uaagaaa:7mer@239, | |
| | | | | | uaagaaa:7mer@852, | |
| | | | | | aagaaa:6mer@1116, | |
| | | | | | aaauga:6mer@1388, | |
| | | | | | uaagaaa:7mer@1969, | |
| | | | | | aaauga:6mer@2202, | |
| | | | | | aagaaa:6mer@2371, | |
| | | | | | uaagaaa:7mer@3889, | |
| | | | | | aaauga:6mer@4160, | |
| | | | | | aagaaa:6mer@4536, | |
| | | | | | aagaaa:6mer@4699, | |
| | | | | | aagaaa:6mer@5249] | |
| | ERG1 | cucauuucuguaguaagac | 1703 | −0.4 | [gaaauga:7mer@181, | 0.32 |
| | | | | | aagaaa:6mer@290, | |
| | | | | | uaagaaa:7mer@303, | |
| | | | | | aagaaa:6mer@749, | |
| | | | | | aaauga:6mer@942] | |
| PCS11 | BCL2 | guuucuuacucagacagag | 2054 | −3.2 | [uaagaaa:7mer@103, | 0.25 |
| | | | | | uaagaaa:7mer@239, | |
| | | | | | uaagaaa:7mer@852, | |
| | | | | | aagaaa:6mer@1116, | |
| | | | | | aaauga:6mer@1388, | |
| | | | | | uaagaaa:7mer@1969, | |
| | | | | | aaauga:6mer@2202, | |
| | | | | | aagaaa:6mer@2371, | |
| | | | | | uaagaaa:7mer@3889, | |
| | | | | | aaauga:6mer@4160, | |
| | | | | | aagaaa:6mer@4536, | |
| | | | | | aagaaa:6mer@4699, | |
| | | | | | aagaaa:6mer@5249] | |
| | ERG2 | cucauuucuguaguaagac | 1745 | −0.4 | [gaaauga:7mer@181, | 0.32 |
| | | | | | aagaaa:6mer@290, | |
| | | | | | uaagaaa:7mer@303, | |
| | | | | | aagaaa:6mer@749, | |
| | | | | | aaauga:6mer@942] | |
| PCS12 | BCL2 | cuuuagaugauaagcauuu | 4659 | 0.0 | [aaagauu:7mer@247, | 0.31 |
| | | | | | ucuaaa:6mer@3138, | |
| | | | | | aagauu:6mer@3271, | |
| | | | | | aagauu:6mer@3306, | |
| | | | | | aucuaaa:7mer@3457, | |
| | | | | | aagauu:6mer@3530, | |
| | | | | | ucuaaa:6mer@4360, | |
| | | | | | aagauu:6mer@5018, | |
| | | | | | aagauu:6mer@5054] | |
| | cMYC | aaaucuuucagucucaaga | 1994 | 0.0 | [aaagauu:7mer@116, | 0.42 |
| | | | | | aagauu:6mer@241] | |
| PCS13 | ETV1 | ccuguuacacagaauaaau | 1726 | 0.0 | [guaacag:7mer@216, | 0.14 |
| | | | | | uuucaa:6mer@1481, | |
| | | | | | auuucaa:7mer@1914, | |
| | | | | | auuucaa:7mer@1940, | |
| | | | | | auuucaa:7mer@3502, | |
| | | | | | uaacag:6mer@3630, | |
| | | | | | uuucaa:6mer@4161] | |
| | cMYC | auugaaauucuguguaacu | 2200 | −0.5 | [uaacag:6mer@26, | 0.25 |
| | | | | | uuucaa:6mer@62, | |
| | | | | | uaacag:6mer@202, | |
| | | | | | auuucaa:7mer@322] | |
| PCS14 | ETV1 | augaaaucaaacagacaug | 1840 | −4.0 | [gcacuu:6mer@169, | 0.28 |
| | | | | | gauuuca:7mer@330, | |
| | | | | | auuuca:6mer@387, | |
| | | | | | auuuca:6mer@782, | |
| | | | | | auuuca:6mer@1190, | |
| | | | | | auuuca:6mer@1914, | |
| | | | | | gauuuca:7mer@1939, | |
| | | | | | agcacuu:7mer@2062, | |
| | | | | | auuuca:6mer@3502, | |
| | | | | | auuuca:6mer@4200, | |
| | | | | | gauuuca:7mer@4364] | |
| | hTERT | caagugcugucugauucca | 1818 | −4.2 | [ ] | −0.07 |
| PCS15 | hTERT | aggccucggccaaacacuc | 3583 | −3.4 | [gaggcc:6mer@115, | 0.17 |
| | | | | | cgaggcc:7mer@137, | |
| | | | | | gaggcc:6mer@176, | |
| | | | | | aaauacu:7mer@523] | |
| | BCL2 | gaguauuugugcagcgagg | 4409 | −3.3 | [aauacu:6mer@833, | −0.15 |
| | | | | | gaggcc:6mer@1001, | |
| | | | | | gaggcc:6mer@1244, | |
| | | | | | aaauacu:7mer@3207, | |
| | | | | | gaggcc:6mer@3222] | |
| PCS16 | ETV1 | aagccauguuuccuguaau | 242 | −0.2 | [uguaaa:6mer@398, | −0.05 |
| | | | | | cuguaaa:7mer@487, | |
| | | | | | uguaaa:6mer@534, | |
| | | | | | cuguaaa:7mer@653, | |
| | | | | | cuguaaa:7mer@1442, | |
| | | | | | uguaaa:6mer@1740, | |
| | | | | | uguaaa:6mer@3754] | |
| | cMYC | auuuacagagaaacauugu | 2139 | −2.6 | [uguaaa:6mer@132, | 0.52 |
| | | | | | cuguaaa:7mer@261, | |
| | | | | | uguaaa:6mer@281] | |
| PCS17 | EGFRa | cugauuucaugacagcaag | 4661 | −0.7 | [gaaauca:7mer@793] | 0.27 |
| | ETV1 | cuuauguuguuaugaaauc | 1851 | −1.3 | [aacauaa:7mer@341, | 0.2 |
| | | | | | aaauca:6mer@749, | |
| | | | | | acauaa:6mer@892, | |
| | | | | | aaauca:6mer@932, | |
| | | | | | aaauca:6mer@2018, | |
| | | | | | acauaa:6mer@2416, | |
| | | | | | gaaauca:7mer@2642, | |
| | | | | | aaauca:6mer@2855, | |
| | | | | | aaauca:6mer@3638, | |
| | | | | | aaauca:6mer@4153, | |
| | | | | | aaauca:6mer@4568] | |
| PCS18 | EGFRa | gccgaagggcaugagcugc | 2615 | −2.9 | [ ] | −0.45 |
| | hTERT | gacacucagcccuuggcug | 3651 | −2.4 | [ugagugu:7mer@125, | −0.25 |
| | | | | | ugagugu:7mer@163, | |
| | | | | | gagugu:6mer@187, | |
| | | | | | ugagugu:7mer@205] | |
| PCS19 | EGFRa | gguggcugccuuucugggc | 3717 | −5.6 | [agccac:6mer@55, | −0.48 |
| | | | | | agccac:6mer@448, | |
| | | | | | agccac:6mer@593, | |
| | | | | | cugggc:6mer@610] | |
| | BCL2 | ggcccagauaggcacccag | 1186 | −7.2 | [cugggc:6mer@1141, | −0.43 |
| | | | | | agccac:6mer@1197, | |
| | | | | | cugggc:6mer@1250, | |
| | | | | | cugggc:6mer@2110] | |
| PCS20 | BCL2 | caaacggagcugcacuuug | 5435 | −1.5 | [ugcauuu:7mer@488, | −0.22 |
| | | | | | gcauuu:6mer@744, | |
| | | | | | ccguuu:6mer@1175, | |
| | | | | | ugcauuu:7mer@2025, | |
| | | | | | gcauuu:6mer@2122, | |
| | | | | | ugcauuu:7mer@2858, | |
| | | | | | uccguuu:7mer@4233, | |
| | | | | | ugcauuu:7mer@4857] | |
| | ERG1 | caaaugcagacuccuguau | 2911 | −0.5 | [ccguuu:6mer@402, | −0.06 |
| | | | | | uccguuu:7mer@556, | |
| | | | | | gcauuu:6mer@1077, | |
| | | | | | ugcauuu:7mer@1194, | |
| | | | | | ugcauuu:7mer@1389] | |
| PCS21 | EGFRa | cuguuaaccagucagguua | 5333 | 0.0 | [guuaaca:7mer@1465, | 0.17 |
| | | | | | caguguu:7mer@1487] | |
| | ERG1 | uaacacugacugcaugaac | 2358 | −1.3 | [caguguu:7mer@836, | 0.32 |
| | | | | | caguguu:7mer@853] | |
| PCS22 | EGFRa | cuguuaaccagucagguua | 5333 | 0.0 | [guuaaca:7mer@1465, | 0.17 |
| | | | | | caguguu:7mer@1487] | |
| | ERG2 | uaacacugacugcaugaac | 2400 | −1.3 | [caguguu:7mer@836, | 0.32 |
| | | | | | caguguu:7mer@853] | |
| PCS23 | hTERT | ucaggccucagacucccag | 3562 | −4.9 | [aggccug:7mer@116, | 0.01 |
| | | | | | aggccug:7mer@139, | |
| | | | | | aggccug:7mer@177] | |
| | cMYC | cucgguguccgaggaccug | 1603 | −5.5 | [ ] | −0.34 |
| PCS24 | cMYC | auaggugauugcucaggac | 1925 | −3.6 | [aaggaa:6mer@8, | 0.25 |
| | | | | | ucaccua:7mer@47] | |
| | ERG1 | guuccuugagccauucacc | 314 | −3.3 | [aaggaa:6mer@121, | 0.27 |
| | | | | | aaggaa:6mer@421, | |
| | | | | | caaggaa:7mer@1499] | |
| PCS25 | hTERT | cagggcacgcacaccaggc | 218 | −6.9 | [ugcccu:6merg355, | −0.27 |
| | | | | | gugcccu:7mer@443, | |
| | | | | | caccugg:7mer@470] | |
| | ERG1 | gccagguguggcguuccgu | 160 | −6.7 | [accugg:6mer@2] | −0.58 |
| PCS26 | ERG2 | acaggagcuccaggaggaa | 1080 | −2.5 | [ugaggag:7mer@185, | −0.38 |
| | | | | | gaggag:6mer@709, | |
| | | | | | gaggag:6mer@730] | |
| | cMYC | ucuccucauggagcaccag | 1280 | −4.6 | [ ] | 0.11 |
| PCS27 | EGFRa | uguugugaaagcauuauca | 5196 | −1.5 | [cacaac:6mer@1228, | 0.41 |
| | | | | | ucacaac:7mer@1328] | |
| | cMYC | ugaucaugcauuugaaaca | 1949 | −2.6 | [caugauc:7mer@71, | 0.21 |
| | | | | | ucacaac:7mer@88] | |
| PCS28 | hTERT | ccaaagagguggcuucuuc | 895 | 0.0 | [ ] | −0.05 |
| | ERG2 | gccaagaaggccaucucuu | 2610 | 0.0 | [ucuugg:6mer@149, | 0.02 |
| | | | | | ucuuug:6mer@930, | |
| | | | | | uucuugg:7mer@1046] | |

### Example 2

The endonuclease Dicer is an important factor in miRNA biogenesis and siRNAs designed as Dicer substrates can have drastically increased potency compared to standard length siRNAs and shRNAs. To explore this possibility, the siRNAs from Example 1 were extended into putative Dicer substrate siRNAs.

Given a list of candidate dsRNAs and their corresponding target sequences, our custom computer program uses a custom module to extend each dsRNA into a Dicer substrate dsRNA (D-dsRNA) with asymmetric 25 and 27 nts long strands. More specifically, the module first arbitrarily assigns one of the two strands to form the 27mer strand. Second, the module adds two and six nts to the strand's 3′ and 5′ ends. These additional nucleotides are the reverse complements of the two and six nts 5′ and 3′ of the strand's cleavage target site. Third, the module adds the reverse complement of the six nucleotides to the 3′ end of the bi-functional siRNA's other strand, such that the resulting strand is 25 nts long. Finally, the module modifies the two nucleotides at the 3′ end of the 25 nts long strand to DNAs instead of RNAs. The program uses the external program RNAhybrid to predict the resulting D-dsRNA's duplex structure.

The D-dsRNAs designed by this method are shown in Table 7 below.

TABLE 7
Dicer substrate siRNAs based on the 19mer duplexes in Table 6. The table
shows the dicer substrate strands and the two strands' duplex as
predicted by RNAhybrid. DNAs are in uppercase whereas
RNAs are in lower case.

| dsRNA | Target | | |
|---|---|---|---|
| Duplex | Gene | Strand Sequences | |
| ID | IDs | 5′ → 3′ | Duplex Structure |
| PCS1 | EGFRa BCL2 | caaacaagucagucuaacgaaauTA uaauuuccuuagaauggcuuguauuuc | 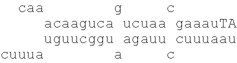 |
| PCS2 | ETV1 BCL2 | auuucucccacgcugacuaccucTC gagagguaagugagcuguggagagaau | 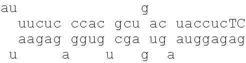 |
| PCS3 | ETV1 BCL2 | uuuaguggaauaguugaaaucuaTT aauagaauuucaacugacucccuaauu | 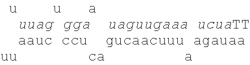 |
| PCS4 | ETV1 cMYC | caauucucuguaucuugcauuaaAT auuuaauggcaauauuuacagagaaac | 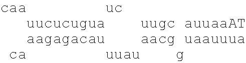 |
| PCS5 | ETV1 ERG1 | guuucaugacuuaguguuuaccaAA uuugguauaacacugacugcaugaacc | 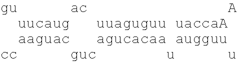 |
| PCS6 | ETV1 ERG2 | guuucaugacuuaguguuuaccaAA uuugguauaacacugacugcaugaacc | 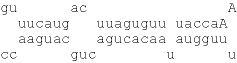 |
| PCS7 | ETV1 ERG1 | ucaaacagacaugauauagcaacTG caguugcauaucaacgucuguugaugg | 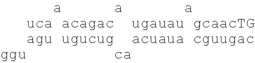 |
| PCS8 | ETV1 ERG2 | ucaaacagacaugauauagcaacTG Caguugcauaucaacgucuguugaugg | 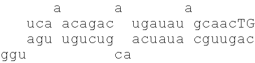 |
| PCS9 | EGFRa ETV1 | aaauucacugcuuuguggcucuuGA ucaagagacagcaaacagcagugaaga | 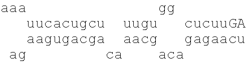 |
| PCS10 | BCL2 ERG1 | guuucuuacucagacagaggaggAT auccuccucauuucuguaguaagacuu | 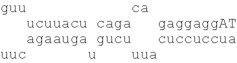 |
| PCS11 | BCL2 ERG2 | guuucuuacucagacagaggaggAT auccuccucauuucuguaguaagacuu | 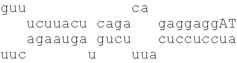 |
| PCS12 | BCL2 cMYC | cuuuagaugauaagcauuuagccAT auggcuaaaucuuucagucucaagacu | 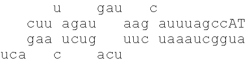 |
| PCS13 | ETV1 cMYC | ccuguuacacagaauaaauccuaGT acuaggauugaaauucuguguaacugc | 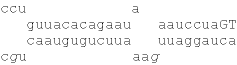 |
| PCS14 | ETV1 hTERT | augaaaucaaacagacaugaagaGG ccucuucaagugcugucugauuccaau | 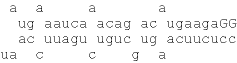 |
| PCS15 | hTERT BCL2 | aggccucggccaaacacuccgcaAG cuugcggaguauuugugcagcgaggga | 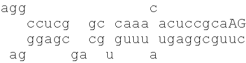 |
| PCS16 | ETV1 cMYC | aagccauguuuccuguaauauugCC ggcaauauuuacagagaaacauugugu | 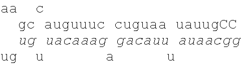 |
| PCS17 | EGFRa ETV1 | cugauuucaugacagcaagauaaTG cauuaucuuauguuguuaugaaaucaa | 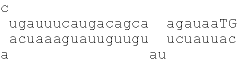 |
| PCS18 | EGFRa hTERT | gccgaagggcaugagcugccagcAC gugcuggacacucagcccuuggcugga | 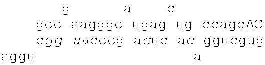 |
| PCS19 | EGFRa BCL2 | gguggcugccuuucugggcacaaGT Acuuguggcccagauaggcacccaggg | 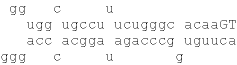 |
| PCS20 | BCL2 ERG1 | caaacggagcugcacuuugcacuTT aaagugcaaaugcagacuccuguauaa | 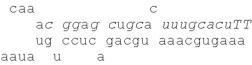 |
| PCS21 | EGFRa ERG1 | cuguuaaccagucagguuauaccAA uugguauaacacugacugcaugaaccc | 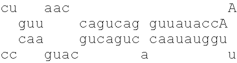 |
| PCS22 | EGFRa ERG2 | cuguuaaccagucagguuauaccAA uugguauaacacugacugcaugaaccc | 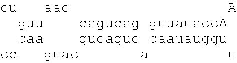 |
| PCS23 | hTERT cMYC | ucaggccucagacucccaggagaAT auucuccucgguguccgaggaccuggg | 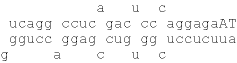 |
| PCS24 | cMYC ERG1 | auaggugauugcucaggacucucCT aggagaguuccuugagccauucaccug | 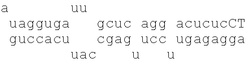 |
| PCS25 | hTERT ERG1 | cagggcacgcacaccaggcuaagAC gucuuagccagguguggcguuccguag | 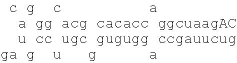 |
| PCS26 | ERG2 cMYC | acaggagcuccaggaggaacaccGC gcggugucuccucauggagcaccaggg | 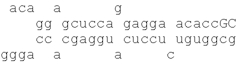 |
| PCS27 | EGFRa cMYC | uguugugaaagcauuaucaaaugCA ugcauuugaucaugcauuugaaacaag | 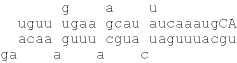 |
| PCS28 | hTERT ERG2 | ccaaagagguggcuucuucugccAC guggcagccaagaaggccaucucuuac | 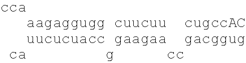 |

### Example 3

When extended to 27mer Dicer substrate dsRNAs (see Example 2), many of the D-dsRNA candidates had an end structure where the predicted number of unpaired bases at the 3′ end of the bottom strand was less than or equal to the predicted number of unpaired bases at the 5′ end of the top strand. Based on the structure of known miRNAs and the binding requirements of the Dicer PAZ-domain, this structure is likely suboptimal for Dicer processing. Therefore the D-dsRNAs that had a duplex end structure that most closely resembles miRNA precursor end structures were manually selected. More specifically, the D-dsRNAs where the number of unpaired bases at the 5′ end of the top strand was less than the number of unpaired bases at the 3′ end of the bottom strand, as predicted by RNAhybrid, were selected.

The selected dsRNAs are shown in Table 8 below. The Table is sorted based on how likely the dsRNAs can cause translational suppression and show the one or two highest-scoring dsRNAs against each possible combination of the target genes.

TABLE 8

Top-scoring multifunctional siRNAs against all combinations of ERG1, MYC,
ERG2, BCL2, hTERT, ETV1, and EGFRa. The table lists dsRNA ID, the target
gene, the corresponding strand, the cleavage location in the target
sequence, the predicted thermodynamic end stability, and the seed site
locations in the target sequence's 3′ UTR. The 3′UTR seed sites
listed in the same row as a target gene are those seed sites found in that
target gene's 3′UTR sequence. A given seed site may be complementary
to the seed region of either strand of the dsRNA. Efficacy is the predicted
cleavage efficacy of a “standard” dsRNA designed against the
corresponding target sites; values >0 indicate effective dsRNAs with larger
values representing more confident predictions. Horizontal lines separate
the different siRNA duplexes. These dsRNAs were selected from the
top-scoring multi-targeting siRNAs (Table 6) based on their duplex end
structure, such that the predicted number of unpaired bases at the
3′ end of the bottom strand is greater than the predicted number
of unpaired bases at the 5′ end of the top strand

| | | | | | | |
|---|---|---|---|---|---|---|
| dsRNA | Target | | | | | |
| Duplex | Gene | Strand Sequence | Cleavage | End | | |
| ID | ID | (5′--> 3′) | Location | Stability | 3′ UTR Seed Sites | Efficacy |

| | | | | | | |
|---|---|---|---|---|---|---|
| PCS1 | EGFRa | caaacaagucagucuaacg | 4600 | −3.7 | [cuuguuu:7mer@732, | 0.05 |
| | | | | | uuguuu:6mer@752] | |
| | BCL2 | ccuuagaauggcuuguauu | 4999 | −2.2 | [uuguuu:6mer@1833, | 0.0 |
| | | | | | uuguuu:6mer@2006, | |
| | | | | | uuguuu:6mer@2046, | |
| | | | | | cuuguuu:7mer@2084, | |
| | | | | | ucuaag:6mer@2840, | |
| | | | | | uuguuu:6mer@3433, | |
| | | | | | uucuaag:7mer@3797, | |
| | | | | | uuguuu:6mer@4892, | |
| | | | | | uuguuu:6mer@4934, | |
| | | | | | uuguuu:6mer@4959, | |
| | | | | | uuguuu:6mer@5182] | |
| PCS3 | ETV1 | uuuaguggaauaguugaaa | 5024 | −2.1 | [cacuaa:6mer@991, | 0.32 |
| | | | | | uugaaa:6mer@1463, | |
| | | | | | uugaaa:6mer@1527, | |
| | | | | | uugaaa:6mer@1557, | |
| | | | | | guugaaa:7mer@2457, | |
| | | | | | uugaaa:6mer@2484, | |
| | | | | | uugaaa:6mer@3415, | |
| | | | | | uugaaa:6mer@3482, | |
| | | | | | ccacuaa:7mer@3514, | |
| | | | | | cacuaa:6mer@3583] | |
| | BCL2 | auuucaacugacucccuaa | 5021 | −1.2 | [cacuaa:6mer@38, | 0.44 |
| | | | | | ccacuaa:7mer@302, | |
| | | | | | uugaaa:6mer@1512, | |
| | | | | | uugaaa:6mer@1684, | |
| | | | | | uugaaa:6mer@2692, | |
| | | | | | guugaaa:7mer@3819, | |
| | | | | | uugaaa:6mer@5000] | |
| PCS7 | ETV1 | ucaaacagacaugauauag | 1834 | −1.6 | [uguuug:6mer@88, | 0.3 |
| | | | | | cuguuug:7mer@324, | |
| | | | | | cuguuug:7mer@563, | |
| | | | | | uugauau:7mer@1032, | |
| | | | | | ugauau:6mer@1341, | |
| | | | | | ugauau:6mer@1410, | |
| | | | | | uguuug:6mer@1460, | |
| | | | | | cuguuug:7mer@2693, | |
| | | | | | uguuug:6mer@3954] | |
| | ERG1 | cauaucaacgucuguugau | 2028 | −1.3 | [uugauau:7mer@506, | 0.09 |
| | | | | | uguuug:6mer@934, | |
| | | | | | ugauau:6mer@968, | |
| | | | | | uguuug:6mer@1512] | |
| PCS12 | BCL2 | cuuuagaugauaagcauuu | 4659 | 0.0 | [aaagauu:7mer@247, | 0.31 |
| | | | | | ucuaaa:6mer@3138, | |
| | | | | | aagauu:6mer@3271, | |
| | | | | | aagauu:6mer@3306, | |
| | | | | | aucuaaa:7mer@3457, | |
| | | | | | aagauu:6mer@3530, | |
| | | | | | ucuaaa:6mer@4360, | |
| | | | | | aagauu:6mer@5018, | |
| | | | | | aagauu:6mer@5054] | |
| | cMYC | aaaucuuucagucucaaga | 1994 | 0.0 | [aaagauu:7mer@116, | 0.42 |
| | | | | | aagauu:6mer@241] | |
| PCS14 | ETV1 | augaaaucaaacagacaug | 1840 | −4.0 | [gcacuu:6mer@169, | 0.28 |
| | | | | | gauuuca:7mer@330, | |
| | | | | | auuuca:6mer@387, | |
| | | | | | auuuca:6mer@782, | |
| | | | | | auuuca:6mer@1190, | |
| | | | | | auuuca:6mer@1914, | |
| | | | | | gauuuca:7mer@1939, | |
| | | | | | agcacuu:7mer@2062, | |
| | | | | | auuuca:6mer@3502, | |
| | | | | | auuuca:6mer@4200, | |
| | | | | | gauuuca:7mer@4364] | |
| | hTERT | caagugcugucugauucca | 1818 | −4.2 | [ ] | −0.07 |
| PCS18 | EGFRa | gccgaagggcaugagcugc | 2615 | −2.9 | [ ] | −0.45 |
| | hTERT | gacacucagcccuuggcug | 3651 | −2.4 | [ugagugu:7mer@125, | −0.25 |
| | | | | | ugagugu:7mer@163, | |
| | | | | | gagugu:6mer@187, | |
| | | | | | ugagugu:7mer@205] | |
| PCS19 | EGFRa | gguggcugccuuucugggc | 3717 | −5.6 | [agccac:6mer@55, | −0.48 |
| | | | | | agccac:6mer@448, | |
| | | | | | agccac:6mer@593, | |
| | | | | | cugggc:6mer@610] | |
| | BCL2 | ggcccagauaggcacccag | 1186 | −7.2 | [cugggc:6mer@1141, | −0.43 |
| | | | | | agccac:6mer@1197, | |
| | | | | | cugggc:6mer@1250, | |
| | | | | | cugggc:6mer@2110] | |
| PCS20 | BCL2 | caaacggagcugcacuuug | 5435 | −1.5 | [ugcauuu:7mer@488, | −0.22 |
| | | | | | gcauuu:6mer@744, | |
| | | | | | ccguuu:6mer@1175, | |
| | | | | | ugcauuu:7mer@2025, | |
| | | | | | gcauuu:6mer@2122, | |
| | | | | | ugcauuu:7mer@2858, | |
| | | | | | uccguuu:7mer@4233, | |
| | | | | | ugcauuu:7mer@4857] | |
| | ERG1 | caaaugcagacuccuguau | 2911 | −0.5 | [ccguuu:6mer@402, | −0.06 |
| | | | | | uccguuu:7mer@556, | |
| | | | | | gcauuu:6mer@1077, | |
| | | | | | ugcauuu:7mer@1194, | |
| | | | | | ugcauuu:7mer@1389] | |

### Example 4

The dsRNAs selected in Example 3 were extended into Dicer-substrate dsRNAs according to the method set out in Example 2. The D-dsRNAs designed by this method are shown in Table 9 below.

TABLE 9
Dicer substrate siRNAs based on the 19mer duplexes in Table 4. The table
shows the dicer substrate strands and the two strands' duplex as
predicted by RNAhybrid. DNAs are in uppercase whereas
RNAs are in lower case.

| dsRNA | Target | | |
|---|---|---|---|
| Duplex | Gene | Strand Sequences | |
| ID | IDs | 5′ → 3′ | Duplex Structure |
| PCS1 | EGFRa BCL2 | caaacaagucagucuaacgaaauTA uaauuuccuuagaauggcuuguauuuc | 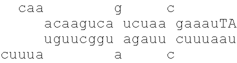 |
| PCS3 | ETV1 BCL2 | uuuaguggaauaguugaaaucuaTT aauagaauuucaacugacucccuaauu | 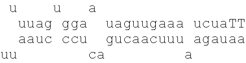 |
| PCS7 | ETV1 ERG1 | ucaaacagacaugauauagcaacTG caguugcauaucaacgucuguugaugg | 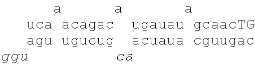 |
| PCS12 | BCL2 cMYC | cuuuagaugauaagcauuuagccAT auggcuaaaucuuucagucucaagacu | 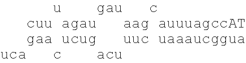 |
| PCS14 | ETV1 hTERT | augaaaucaaacagacaugaagaGG ccucuucaagugcugucugauuccaau | 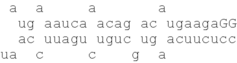 |
| PCS18 | EGFRa hTERT | gccgaagggcaugagcugccagcAC gugcuggacacucagcccuuggcugga | 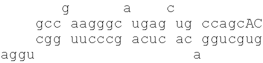 |
| PCS19 | EGFRa BCL2 | gguggcugccuuucugggcacaaGT acuuguggcccagauaggcacccaggg | 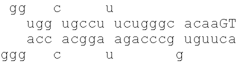 |
| PCS20 | BCL2 ERG1 | caaacggagcugcacuuugcacuTT aaagugcaaaugcagacuccuguauaa | 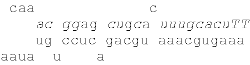 |

### Example 5

Standard edit distance allows for insertions and deletions in addition to substitutions. Consequently, when using standard edit distance to identify dsRNA candidates in step (ii) of the design algorithm, the resulting dsRNAs can become somewhat asymmetric; that is, the predicted duplexes may contain asymmetric bulges and internal loops.

Therefore, as an alternative to the standard edit distance, an option was introduced in our design program that restricted step (ii) in the algorithm to only allow substitutions in the edit distance computation. Running this modified version of the program, but still requiring a maximum edit distance of five (substitutions) identified a set of dsRNAs with slightly different predicted duplex structures. Three of these alternative dsRNAs are shown in Table 10 below:

TABLE 10
dsRNAs designed by only allowing substitutions
during the edit distance screen as described
above. The table lists the dsRNA ID, the target
gene IDs and the 5′ to 3′ sequences
of both strands of the dsRNA

| | Strand 1 | | Strand 2 | |
|---|---|---|---|---|
| | | Strand | | Strand |
| dsRNA | Target | Sequence | Target | Sequence |
| Duplex ID | Gene ID | 5′ -> 3′ | Gene ID | 5′ -> 3′ |
| PCS29 | ETV1 | aucuugagaug | cMyc | aaucuuucagu |
| | | gagagauu | | cucaagac |
| PCS30 | EGFRa | gaauagaugac | ETV1 | ugguuugaguc |
| | | ucaaggca | | aucuauuu |
| PCS31 | cMyc | caaguccucuu | ERG1 | cauuucuuaag |
| | | cagaaaug | | accacuuu |

### Example 6

The dsRNAs designed in Example 5 were extended into Dicer-substrate dsRNAs according to the method set out in Example 2. The D-dsRNAs designed by this method are shown in Table 11 below:

TABLE 11
Dicer substrate dsRNAs for dsRNAs from Table 10. The table shows the
dicer substrate strands and the two strands' duplex as predicted by
RNAhybrid. DNAs are in uppercase whereas RNAs
are in lower case.

| dsRNA | Target | | |
|---|---|---|---|
| Duplex | Gene | Strand Sequences | |
| ID | IDs | 5′ → 3′ | Duplex Structure |
| sPCS29 | ETV1 cMyc | aucuugagauggagagauuuagcCA uggcuaaaucuuucagucucaagacuc | 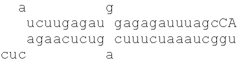 |
| sPCS30 | EGFRa ETV1 | gaauagaugacucaaggcauuuaTT aauaaaugguuugagucaucuauuuug | 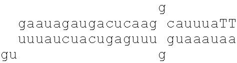 |
| sPCS31 | cMyc ERG1 | caaguccucuucagaaauguauaAA uuuauacauuucuuaagaccacuuucu | 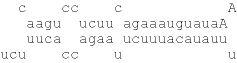 |

What is claimed is:

1. A double-stranded RNA molecule in which each strand of said molecule possesses:
(a) sufficient complementarity to a target mRNA molecule to facilitate cleavage thereof; and
(b) sufficient complementarity to the other strand of the double-stranded RNA molecule so as to form a stable duplex;
and in which at least one strand of said molecule possesses:
(c) a seed region of complementarity to at least one seed site present in a 3′ untranslated region of at least one target mRNA molecule.

2. The double-stranded RNA molecule of claim 1 wherein each strand of the duplex is at least 12 nucleotides in length.

3. (canceled)

4. The double-stranded RNA molecule of claim 1 in which one strand of the duplex is 25 nucleotides in length and the other strand of the duplex is 27 nucleotides in length.

5. (canceled)

6. The double-stranded RNA molecule of claim 1 wherein each strand of the duplex possesses no more than 5 mismatches with a region of the target mRNA which is to be cleaved.

7. The double-stranded RNA molecule of claim 1 wherein the seed region possesses no more than 3 mismatches with the seed site(s) of the target mRNA(s).

8. The double-stranded RNA molecule of claim 1 wherein there are at most 5 base pair mismatches between the two strands of the duplex.

9. (canceled)

10. The double-stranded RNA molecule of claim 1 wherein at least one strand of the duplex possesses a seed region of complementarity to a seed site present in a 3′ untranslated region of a target mRNA molecule to which said strand also has sufficient complementarity to facilitate cleavage thereof.

11. The double-stranded RNA molecule of claim 1 wherein at least one strand of the duplex possesses a seed region of complementarity to a seed site present in a 3′ untranslated region of a target mRNA molecule which is different to the target mRNA molecule to which said strand of the duplex has sufficient complementarity to facilitate cleavage thereof.

12. The double-stranded RNA molecule of claim 1 wherein both strands of the duplex possess a seed region of complementarity to at least one seed site present in a 3′ untranslated region of at least one target mRNA molecule.

13. The double-stranded RNA molecule of claim 1 wherein strand 1 and strand 2 thereof have the sequences set out in Table 2 and Table 3.

14. A single-stranded RNA molecule comprising a sequence selected from the sequences set out in Table 2 and Table 3.

15. An algorithm for the design of a double-stranded RNA molecule in which each strand of said molecule possesses:
(a) sufficient complementarity to a target mRNA molecule to facilitate cleavage thereof; and
(b) sufficient complementarity to the other strand of the double-stranded RNA molecule so as to form a stable duplex;
and in which at least one strand of said molecule possesses:
(c) a seed region of complementarity to at least one seed site present in a 3′ untranslated region of at least one target mRNA molecule;
wherein said algorithm comprises the steps:
(i) input a population of mRNA sequences transcribed from one or more genes of interest;
(ii) identify all subsequences of at least 12 nucleotides in length within the population of step (i) which are complementary to another subsequence of at least 12 nucleotides in length in the population;
(iii) determine a list of candidate bi-functional double-stranded RNA molecules, said list comprising the double-strand RNA duplexes comprising the two complementary subsequences of step (ii); and
(iv) sort the list of candidate double-stranded RNA molecules of step (iii) based on their potential to cause translational suppression.

16. The algorithm of claim 15, wherein the algorithm comprises an additional step following step (iii) or (iv) as follows:
from the list of candidate bi-functional double-stranded RNA molecules, remove all double-stranded RNA molecules that:
(p) contain one or more of the motifs aaaa, cccc, gggg, or tttt in either strand; and/or
(q) have a GC-percentage less than 25% or greater than 75% in either strand; and/or
(r) have a high probability of cleavage-based off-target effects; and/or
(s) have a large difference in duplex thermodynamic end stability.

17. The algorithm of claim 15 wherein step (iv) comprises the following steps:
(t) identify the target mRNAs' seed sites complementary to the seed region of one or both strands of the double-stranded RNA molecule;
(u) structure the seed sites into seed site modules; and
(v) score individual seed site modules to identify the module with the highest regulatory potential.

18.-25. (canceled)

26. A method of designing a double-stranded RNA molecule which comprises performing the algorithm of claim 15.

27. A method of producing a double-stranded RNA molecule which comprises performing the algorithm of claim 15 and then synthesising one or more of the double-stranded RNA molecules generated by said algorithm.

28.-31. (canceled)

32. A method of treating cancer and cancer related diseases comprising administration of an effective amount of the double-stranded RNA molecule of claim 1.

33. (canceled)

34. A pharmaceutical composition comprising the double-stranded RNA molecule of claim 1 and a physiologically acceptable carrier, diluent or excipient.

35. A kit or administration device comprising the double-stranded RNA molecule of claim 1 and information material which describes administering the double-stranded RNA molecule to a human or other animal.

36. The double-stranded RNA molecule of claim 1 wherein the target mRNA molecule is transcribed from a gene implicated in disease.
